(19) United States
(12) Patent Application Publication
(10) Pub. No.: US 2022/0251632 A1
(43) Pub. Date: Aug. 11, 2022

(54) METHOD FOR SPATIALLY BARCODING CELLS IN TISSUE SLICES

(71) Applicant: University of Washington, Seattle, WA (US)

(72) Inventors: Mary Regier, Seattle, WA (US); Kelly Rose Stevens, Seattle, WA (US); Sanjay Srivatsan, Seattle, WA (US); Cole Trapnell, Seattle, WA (US)

(73) Assignee: University of Washington, Seattle, WA (US)

(21) Appl. No.: 17/629,255

(22) PCT Filed: Jul. 22, 2020
(86) PCT No.: PCT/US2020/043117

§ 371 Date: Jan. 21, 2022

Related U.S. Application Data
(60) Provisional application No. 63/013,379, filed on Apr. 21, 2020.
(60) Provisional application No. 62/877,615, filed on Jul. 23, 2019.

(51) Int. Cl. C12Q 1/6816; C12Q 1/6806; C12Q 1/6876; G01N 33/533

(52) U.S. Cl. CPC C12Q 1/6816; G01N 33/533; C12Q 1/6876; C12Q 1/6806

(57) ABSTRACT

Substrates and methods for spatially resolved analyses of tissue samples are provided, allowing the data from such analyses to be mapped back to the tissue's initial architecture. For analyses that have previously been conducted on bulk, homogenized samples or on dissociated cell components not directly traceable to their prior positions in the tissue sample, spatial resolution of this data can allow better characterization of cell-cell and cell-microenvironment relationships and interactions. This spatial context can aid in determining structure-function relationships in healthy and diseased tissues may thereby enhance the scientific understanding of tissue homeostasis, development, disease, and repair. The information determined using the methods of the disclosure can also be applied to enhancing diagnostics evaluation of diseased tissues.

### CROSS REFERENCE TO RELATED APPLICATIONS

This application claims the benefit of U.S. Provisional Application No. 62/877,615 filed Jul. 23, 2019 and U.S. Provisional Application No. 63/013,379 filed Apr. 21, 2020, expressly incorporated hereby in their entireties.

### STATEMENT OF GOVERNMENT LICENSE RIGHTS

This invention was made with government support under Grant Nos. HD088158, HL137188, and U54 HL145611, awarded by the National Institutes of Health. The government has certain rights in the invention.

### BACKGROUND

Resolving the contributions of spatial context to cellular heterogeneity is a major challenge when identifying links between tissue, cellular and molecular processes. These links underlie crucial processes in biology including development, disease, and homeostasis. Currently, established methods allow for a limited number of readouts, while simultaneously preserving spatial context between groups of cells (e.g. in tissue samples and slices). Limitations with these techniques include but are not limited to the number of distinct molecules assayed (e.g. constraints on fluorescence channels for imaging), scale of spatial resolution (sampling large areas together in bulk analyses), and throughput (time consuming procedures). Recently developed methods measure cellular components in conjunction with spatial position by deploying one of three different strategies. 1) Cells are lysed in tissue slices followed by the in situ capture of the tissue constituents (Stihl et al, 2016; Salmén et al, 2018; Vickovic et al, 2019; Rodriquez et al, 2019). 2) Spatial information is inferred and assigned to dissociated components (Satija et al, 2015). 3) The assay is conducted in situ on a given tissue sample (Lee et al, 2014; Chen et al, 2015; Cornett et al, 2007; Merritt et al, 2019; Wang et al, 2018).

A need exists for improved methods of spatially resolved tissue sample analysis to address these limitations. A need exists for tools allowing spatially resolved tissue analysis. The present invention fulfills this need and provides further related advantages.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a substrate for spatially barcoding a tissue sample, comprising a matrix, wherein the matrix comprises a plurality of discrete labeling regions arranged in a pattern, wherein the labeling regions are separated by non-labeling regions, and wherein each labeling region comprises one or more barcode labels, wherein the one or more barcode labels can be transferred from the matrix into a tissue sample upon contacting the substrate with the tissue sample. In some embodiments, the one or more barcode labels are configured to be transferred into the tissue sample upon contacting the substrate with the tissue sample.

In some embodiments, the matrix further comprises a plurality of discrete reference regions, wherein each reference region comprises one or more reference labels, wherein the one or more reference labels can be transferred from the matrix into a tissue sample upon contacting the substrate with the tissue sample. In some embodiments, the one or more reference labels serve as waypoints or identifiers of a particular sector of the substrate. In some embodiments, the at least a portion of the labeling regions are reference regions.

In some embodiments, the matrix comprises a natural or synthetic polymer. In some embodiments, the matrix comprises agarose.

In some embodiments, the one or more barcode labels and/or the one or more reference labels are fluorescent or non-fluorescent. In some embodiments, the one or more reference labels comprise one or more fluorescent dyes.

In some embodiments, the one or more barcode labels and/or the one or more reference labels comprise one or more staining dyes.

In some embodiments, the staining dye is a dye targeting one or more cellular compartments selected from cell membrane, adiposomes, cytoskeleton, endoplasmic reticulum, endosomes, golgi complexes, lysosomes, mitochondria, nucleus/nucleolus, nuclear membrane, peroxisomes, or combinations thereof. In some embodiments, the staining dye is a lipophilic tracer, wheat germ agglutinin, soybean agglutinin, phalloidin conjugate, paclitaxel conjugate, docetaxel conjugate, DAPI, SYBR green, or Hoechst 33342.

In some embodiments, the one or more barcode labels comprises a molecular tag encoding spatial coordinates within the matrix. In some embodiments, the molecular tag is an antibody, an antibody fragment, or a polynucleotide. In some embodiments, the at least a portion of the labeling regions each comprise a unique combination of the one or more barcode labels. In some embodiments, each of the labeling regions comprises a unique combination of the one or more barcode labels.

In some embodiments, the labeling regions and/or reference regions are prepared by spotting droplets of a solution comprising the one or more barcode labels onto the matrix. In some embodiments, the droplet has a volume of about 1 nL, about 0.5 nL, about 0.4 nL, about 0.3 nL, about 0.2 nL, about 0.1 nL, about 50 pL, about 20 pL, or about 10 pL.

In some embodiments, the labeling regions have a distance of about 250 μm, about 220 μm, about 200 μm, about 190 μm, about 180 μm, about 170 μm, about 160 μm, about 150 μm, about 140 μm, about 130 μm, about 120 μm, about 110 μm, about 100 μm, or about 50 μm between the centers of the labeling regions. In some embodiments, the labeling regions have a diameter of from 0.5 μm to 25 μm, from 1 μm to 200 μm, or from 50 μm to 200 μm.

In some embodiments, the labeling regions are arranged in a grid.

In some embodiments, the substrate comprises about 3,000, about 4,000, about 5,000, about 6,000, about 7,000, about 8,000, about 9,000, about 10,000, about 25,000, about 50,000, about 100,000, about 200,000, about 300,000, about 400,000, about 500,000, about 600,000, about 700,000, about 800,000, about 900,000, or about 1,000,000 unique labeling regions in a 10 mm by 10 mm area.

In some embodiments, the substrate further comprises a support. In some embodiments, the support comprises glass or synthetic polymer.

In another aspect, the disclosure provides a method of spatially labeling cells in a tissue sample, comprising contacting a tissue sample comprising a plurality of cells with the substrate of any one of Claims 1-23 for a time sufficient for at least a portion of the one or more barcode labels to transfer into at least a portion of the plurality of cells.

In some embodiments, the tissue sample is a tissue slice, a cell culture sample, or a biopsy. In some embodiments, the tissue sample is a tissue slice or tissue biopsy. In some embodiments, the tissue sample is fresh, frozen, or paraffin-embedded tissue. In some embodiments, the tissue sample comprises one or more types of cells.

In some embodiments, the time sufficient for at least a portion of the one or more barcode labels to transfer into at least a portion of the plurality of cells is about 1 minute, about 2 minutes, about 3 minutes, about 4 minutes, about 5 minutes, from about 1 minute to about 10 minutes, or from about 3 minutes to about 5 minutes.

In some embodiments, the method further comprises dissociating the tissue sample into individual cells after contacting the sample with the substrate.

In some embodiments, the tissue sample is sandwiched between a glass slide or plastic slide and the substrate during the contacting.

In another aspect, the disclosure provides a kit for spatially labeling cells in a multi-cellular sample, comprising a substrate disclosed herein.

In some embodiments, the kit further comprises means for securing a multi-cellular sample to the substrate and one or more optional components such as buffers.

In another aspect, provided herein is a method of spatial gene expression profiling in a multi-cellular sample, comprising:

(a) contacting a tissue sample comprising a plurality of cells with a substrate of the disclosure for a time sufficient for at least a portion of the one or more barcode labels to transfer into at least a portion of the plurality of cells;

(b) imaging the substrate to determine positions of the reference regions;

(c) extracting nuclei from each cell; and

(d) analyzing gene expression in each individual cell.

In some embodiments, the method comprises dissociating the tissue sample into individual cells after imaging the substrate. In some embodiments, the tissue sample is a tissue slice or tissue biopsy. In some embodiments, the tissue sample is sandwiched between a glass slide or plastic slide and the substrate during the contacting. In some embodiments, the contacting is done in a buffer allowing diffusion of the barcode labels to the cells. In some embodiments, the tissue sample is pre-treated with a permeabilizing agent prior to or during contacting with the substrate.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1C demonstrate that an exemplary substrate and method, referred to as sci-Space, recovers single cell transcriptomes while recording spatial coordinates. Fresh-frozen, sectioned tissues are exposed to a grid of arrayed single-stranded oligos, a subset of which are fluorescent waypoints, and then imaged (1A). The oligo transfer and imaging procedure takes 3 to 5 minutes per slide. The cells from each slide are labeled with an additional, section-identifying barcode prior to pooling and sci-RNA-seq. Joint embedding of E14.0 single cell transcriptomes from this study and published data from single cell transcriptomes spanning E9.5 to E13.5. Major trajectories are labeled. (1B) UMAP embedding of cells from sectioned E14.0 mouse embryos. The most abundant cell types are annotated. (1C)

FIGS. 2A-2F demonstrate that exemplary method and using exemplary substrate, sci-Space, captures spatially and cell type-resolved gene expression across the embryo. Co-registered DAPI stained section image and oligo array, superimposed. SYBR waypoints are highlighted. (2A) Anatomical regions of Slide 1 (left) and an adjacent immunostained serial section aligned to Slide 1 (right). (2B) Highlighted cell types mapping to a single slide. (2C) UMAP embedding as in FIG. 1C but colored by anatomical regions. (2D) Gene expression of dopamine transporter Slc6a3 from sci-Space data (left) and published section/stage matched in situ (right). (2E) Smoothed percentage of gene expression for Igf2 (left) and Myh6 (right) in either cardiomyocytes (top) or endothelial cells (bottom). Color scaled to maximum percentage within each gene. (2F) FIGS. 3A-3M show spatially restricted gene expression of the developing spinal cord. Number of spatially significant (FDR<0.001) auto-correlated genes within each slide (color) and cell type. (3A) Only cell types with more than 50 cells per slide were included. (3B) Log-log (natural log) plot of autocorrelation in UMAP embedding (x-axis) versus auto-correlation in spatial coordinate (y-axis) for each gene. Computed on excitatory neurons from Slide 1. Moran's I values close to 1 indicate perfect spatial correlation, while a value of 0 indicates a random spatial distribution. Selected genes in different regimes highlighted. c-e, Gene expression for three genes from FIG. 3B (rows) visualized by spatial position (left), UMAP (middle) and the percentage of cells expressing that gene in each UMAP excitatory neuron subcluster (right) with an inset of UMAP shown. Left and middle columns indicate expression levels. (3F) Expression patterns of other spatially restricted genes that are not restricted to an excitatory neuron subcluster. (3G) Similar to 3B with Hox genes highlighted. (3H) Natural log-scale boxplot of Moran's I statistic for Hox genes displayed in 3B versus all other genes (p-value<0.001). Normalized gene expression of HoxC cluster in Slide 1. (3I) Row centered and scaled gene expression values for 150 genes (columns) which varied as a function of AP position (rows) across the spinal cord. (3J) All non-Hox gene transcription factors are labelled. Co-embedding of E13.5 cells from the spinal cord atlas and spinal cord cells from sci-Space dataset. Principal graph is shown over embedding. (3K).

FIGS. 4A-4D demonstrates quantifying the variance in gene expression attributable to spatial position. Pairwise angular distance (radians) between global transcriptomes of cells of the indicated cell types. Cell pairs are grouped by physical distance (mm). *** indicates p value<0.0001, Wald linear regression test. (4A) Proportion of variance, apart from that attributable to sparse UMI sampling, explained within cells of each type by spatial position. Points indicate independent estimates from each slide. (4B) UMAP embedding displaying co-embedding of endothelial cells from sci-Space and MOCA datasets. Cells are colored by anatomical annotation (above) or the embryonic stage from which they derive (below) (4C) Smoothed (kriged) aggregate expression of genes in selected spatially autocorrelated gene modules. Colors scaled independently for each module. (4D)

FIGS. 5A-5E show an exemplary substrate with a space-grid design. Schematic of spotted oligos with SYBR green fluorescent dye marked positions labeled in black, each position contains poly-adenylated single stranded DNA. (5A) Overlay of brightfield and fluorescence image of the same position. (5B) Average radius and spot-to-spot distance computed from imaged slides. (5C) Diagram of hierarchical barcoding approach where each position is marked by a unique combination of one of 16 sector barcodes (colors) and one of 1536 spot sequences. (5D-E) An example displaying a single spot oligo barcode (white square) which is in 5 different sectors (5E).

FIGS. 6A-6C show SYBR green waypoints transfer to DAPI-stained embryo. Permeabilized mouse embryo section (6A) receives SYBR green waypoints spotted at a single section. (6B) The resulting transfer and imaging shows the location of each waypoint on the DAPI stained section. (6C) Dashed white lines denote the approximate location of each sector.

FIGS. 7A-7G demonstrate that labeling of cryosectioned tissues with barcode labels (referred to as hash oligos) from an agarose coated exemplary substrate is compatible with sci-RNA-seq. Slides from sections of the developing mouse embryo were first labeled with a slide specific oligo and then labeled with another hash oligo from a space-grid containing a single hash oligo at varying concentrations. Replicates are independent experiments performed on different days using tissue sections from a single batch. 7A: RNA UMIs recovered per cell across stage and replicates. 7B: Number of cells sampled from each slide across stages and replicates. 7C-7E: UMAP embedding colored by (7C) embryonic stage, (7D) replicate or (7E) expression of skeletal muscle marker Titin (Ttn). 7F: Correlation of RNA UMIs recovered per gene between replicates at different stages. 7G: Hash UMIs recovered per cell of oligo spotted at 10 μM, 20 μM, 25 μM and 50 μM concentrations.

FIGS. 8A-8C show that exemplary spotted space-grids are reproducible. 8A: HEK293T nuclei were exposed to hash-oligos dissolved from one of 3 space-grids. 8B: Correlation between spot oligo counts originating from different slides. 8C: Distribution of sector oligos observed per cell, broken out by replicate.

FIGS. 9A-9C show exemplary configuration for transfer of space-grid oligos to the nuclei of fresh frozen cryosections. 9A: Spatially indexed slides, “space-grids,” were fabricated by spotting unique combinations of hashing oligos onto agarose membrane-coated slides. 9B: Permeabilized fresh-frozen tissue sections (9C) received the spatially-defined pattern of oligos by diffusion from the space-grids when the oligo-laden agarose and tissue section were sandwiched together between their carrier slides.

FIGS. 10A-10E show exemplary substrate and method (referred herein as sci-Space) workflow for sequencing library preparation and demultiplexing transcripts allows transcripts and spatial positions to be assigned to individual nuclei. 10A: Hashing oligos or barcodes are transferred to nuclei as determined by nuclei positions relative to the barcode array. 10B: Nuclei from each slide are dissociated and labelled with an additional slide-specific barcode. 10C: Transcripts and barcodes are tagged with nuclei-specific indices according to the sci-RNAseq protocol. 10D: Barcodes and transcripts from all nuclei are pooled and sequenced. 10E: Indices are used to demultiplex transcripts and barcodes, which allow for the assignment of each nucleus to its slide, sector, and spot of origin.

FIGS. 11A-11D show co-registration procedure of imaged section and space-grid. 11A: DAPI stained E14 section (Slide 3) with SYBR green points imaged in the GFP-channel. Matched SYBR green waypoints between the image and (11B) the intended SYBR pattern on an ideal space-grid are used to calculate an affine-transformation. 11C: Co-registered imaging data with inferred positions overlaid with image with inset highlighted (11D).

FIG. 12 depicts an exemplary clip.

### DETAILED DESCRIPTION

The disclosure provides substrates and methods for spatially resolved analyses of tissue samples by allowing the components of the sample to be dissociated and analyzed separately in parallel and for the data from those analyses to be mapped back to the tissue's initial architecture. For analyses that have previously been conducted on bulk, homogenized samples or on dissociated cell components not directly traceable to their prior positions in the tissue sample, spatial resolution of this data can contain added contextual information informative in better characterizing cell-cell and cell-microenvironment relationships and interactions. This spatial context can aid in determining structure-function relationships in healthy and diseased tissues and can thereby enhance the scientific understanding of tissue homeostasis, development, disease, and repair. This information can also be applied to enhancing diagnostics evaluation of diseased tissues.

Accordingly, in one aspect, the disclosure provides a substrate for spatially barcoding a multi-cellular tissue sample comprising a matrix, wherein the matrix comprises a plurality of labeling regions arranged in a pattern, wherein the labeling regions are separated by non-labeling regions, and wherein each labeling region comprises one or more barcode labels, wherein the one or more barcode labels can be transferred/are transferrable, e.g., by diffusion, from the matrix into a tissue sample upon contacting the substrate with the tissue sample. In some embodiments, at least a portion of the labeling regions are surrounded by non-labeling regions. In some embodiments, at least a portion of the labeling regions are located directly adjacent to one or more different labeling region.

In some embodiments, at least a portion of the labeling regions comprise a unique combination of the one or more barcode labels. In some embodiments, all labeling regions comprise a unique combination of the one or more barcode labels, so that no labeling regions of the substrate comprise the same combination of the one or more barcode labels.

In some embodiments of the substrates disclosed herein, the matrix further comprises a plurality of discrete reference regions, wherein each reference region comprises one or more reference labels. In some embodiments, the one or more reference labels can be transferred/are transferrable from the matrix into a tissue sample upon contacting the substrate with the tissue sample. The reference regions of the substrates can provide waypoints and/or identifiers of a particular segment or position of the substrate.

In some embodiments, at least a portion of the labeling regions are also reference regions, i.e., these regions can comprise a plurality of the one or more barcode labels and the one or more reference labels. In some embodiments, at least a portion of the labeling regions is about 1%, about 2%, about 3%, about 5%, or about 10% of the labeling regions. In some embodiments, the labeling regions and reference regions can overlap, e.g., completely or partially.

Any suitable synthetic or natural matrix can be used in the substrates of the disclosure. In some embodiments, the matrix comprises a natural or synthetic polymer, such as a hydrogel. In some embodiments, the matrix comprises a polymer with an ethylenic backbone, such as polyacrylamide. In some embodiments, the matrix comprises a carbohydrate hydrogel. In some embodiments, the matrix comprises an agarose gel. In some embodiments, the matrix is an agarose membrane.

The matrix of the substrates of the disclosure can have any suitable thickness. For example, in some embodiments, the matrix can comprise a membrane having a thickness from about 0.01 mm to about 1 mm. In some embodiments, the matrix is prepared by casting an agarose gel having a thickness from about 0.1 mm to about 0.5 mm on top of a support, such as glass, and subsequently drying the gel.

The barcode labels used in the substrates and methods disclosed herein can be labels detectable or identifiable by any suitable methods or a combination of methods, for example, genetic sequencing, mass spectrometry, fluorescence spectroscopy, fluorescence imaging, etc. In some embodiments, the barcode labels can be fluorescent or non-fluorescent.

In some embodiments, the barcode labels and/or reference labels are fluorescent and/or comprise one or more fluorescent dyes. In certain embodiments, the barcode labels comprise a staining dye. Any histology staining dye can be used in the substrates or methods disclosed herein; for example, the staining dye is a dye targeting one or more cellular compartments selected from cell membrane, adiposomes, cytoskeleton, endoplasmic reticulum, endosomes, golgi complexes, lysosomes, mitochondria, nucleus/nucleolus, nuclear membrane, peroxisomes, or combinations thereof. In some embodiments, the staining dye is a lipophilic tracer (e.g., DiI, DiO, DiD, DiA, and DiR; a lectin such as wheat germ agglutinin or soybean agglutinin), a cytoskeleton-specific dye (e.g., a phalloidin conjugate), a tubulin-specific dye (e.g., a paclitaxel conjugate or a docetaxel conjugate), or a nucleus-specific dye (e.g., DAPI, SYBR green, or Hoechst 33342).

In some embodiments, the one or more labeling regions and/or one or more reference regions can comprise a molecular tag encoding spatial coordinates within the matrix. The molecular tag can be an antibody, an antibody fragment, or a polynucleotide. In some embodiments, the substrates or matrices comprise two or more types of labeling regions, wherein each region comprises a distinct barcode label or a distinct combination of barcode labels.

In some embodiments, the one or more barcode labels and/or one or more reference regions comprise an antibody, an antibody fragment, an antibody-oligonucleotide conjugate, or an oligonucleotide, each of which can be unlabeled or labeled with one or more fluorescent dyes or another traceable moiety. In some embodiments, the barcode labels are oligonucleotides, also referred to herein as oligonucleotide barcode labels or hashing oligonucleotides. Any suitable oligonucleotides can be used as barcode labels of the disclosure. In some embodiments, the oligonucleotides are unmodified DNA or RNA oligonucleotides. In some embodiments, each barcoding region comprises a unique combination of one or more oligonucleotide barcode labels. The oligonucleotide barcode labels typically comprise from about 50 to about 200 nucleotides. In some embodiments, the oligonucleotide barcode labels are polyadenylated. In some embodiments, the oligonucleotide barcode labels comprise a 3-polyadenosine segment. In some embodiments, the oligonucleotide barcode labels comprise a segment comprising about 10 to about 50 adenosine nucleotides at the 3′ end. In some embodiments, each oligonucleotide barcode label comprises a unique sequence. In some embodiments, the oligonucleotide barcode labels have a sequence that is not naturally present in the tissue sample to be analyzed. In some embodiments, the oligonucleotide labels comprise a primer sequence. In some embodiments, the oligonucleotide labels comprise barcode or hashing sequence comprising about 10 to about 50 nucleotides. In some embodiments, the oligonucleotide barcode labels comprise a primer sequence comprising about 20 to about 50 nucleotides, a barcode or hashing sequence comprising about 10 to about 50 nucleotides, and a polyadenosine segment comprising about 10 to about 50 adenosine nucleotides. Any suitable primer sequence can be used in the oligonucleotide barcode labels of the disclosure. Examples of oligonucleotide barcode labels are known in the art and include those described in Srivatsan, S. R. et al. Massively multiplex chemical transcriptomics at single-cell resolution. Science 367, 45-51 (2020), the disclosure of which is incorporated herein by reference. In some embodiments, an exemplary barcode can be represented by the formula:

5′-GTCTCGTGGGCTCGGAGATGTGTATAAGAGACAG-[10-
nucleotide barcode sequence]-BAAAAAAAAAAAAAAAAAAA
AAAAAAAAAAAAA-3′,

wherein B denotes G, C or T.

In some embodiments, the one or more reference labels comprises a staining dye, e.g., a fluorescent dye. Any suitable dye can be incorporated into the reference regions. The staining dye can be a dye targeting one or more cellular compartments selected from cell membrane, adiposomes, cytoskeleton, endoplasmic reticulum, endosomes, golgi complexes, lysosomes, mitochondria, nucleus/nucleolus, nuclear membrane, peroxisomes, or combinations thereof. In some embodiments, the staining dye can be a lipophilic tracer, wheat germ agglutinin, soybean agglutinin, phalloidin conjugate, paclitaxel conjugate, docetaxel conjugate, DAPI, SYBR green, Hoechst 33342, or any of the other dyes described herein.

The substrates of the disclosure can be prepared by any suitable method. In some embodiments, the labeling regions are formed by spotting droplets of a solution, e.g., aqueous solution, comprising one or more barcode labels (or one or more barcode labels and reference labels) onto the matrix. Any suitable means for depositing the droplets can be used herein, including but not limited to liquid-handling robots, microarray spotting instruments, and ink jet printers. Typically, the droplets used for depositing the barcode labels onto the matrix have a volume of about 1 nL, about 0.5 nL, about 0.4 nL, about 0.3 nL, about 0.2 nL, about 0.1 nL, about 50 pL, about 20 pL, or about 10 pL.

In some embodiments, the labeling regions have a distance of about 250 μm, about 220 μm, about 200 μm, about 190 μm, about 180 μm, about 170 μm, about 160 μm, about 150 μm, about 140 μm, about 130 μm, about 120 μm, about 110 μm, about 100 μm, or about 50 μm between the centers of the labeling regions. In certain embodiments, the labeling regions have a diameter of from 0.5 μm to 25 μm, from 1 μm to 200 μm, or from 50 μm to 200 μm. The labeling areas can have any suitable shape. In some embodiments, the labeling regions are circular.

The substrates disclosed herein can comprise any suitable number of labeling regions. In some embodiments, the substrate comprises about 3,000, about 4,000, about 5,000, about 6,000, about 7,000, about 8,000, about 9,000, about 10,000, about 25,000, about 50,000, about 60,000 about 70,000, about 80,000, about 90,000, about 100,000, about 200,000, about 300,000, about 400,000, about 500,000, about 600,000, about 700,000, about 800,000, about 900,000, or about 1,000,000 unique labeling regions in a 10 mm by 10 mm area.

The labeling regions and/or reference regions can be arranged in any suitable manner. In some embodiments, the labeling regions can be arranged in a pattern. In some embodiments, the labeling regions can be arranged in a regular or repeating pattern. In certain embodiments, the labeling regions are arranged in a grid pattern. In some embodiments, the labeling regions can be arranged in a hexagonal, e.g. “cannonball packing,” pattern.

The substrates disclosed herein can further comprise a support which can comprise any suitable material, for example, glass, paper, natural polymer, or synthetic polymer. In some embodiments, the support is glass, such as a microscope slide or cover glass. In some embodiments, the support is a glass sheet. The substrates of the disclosure can have any suitable shape. In some embodiments, the substrates are rectangular. In some embodiments, the substrates are configured to be used with known imaging instruments, e.g., fluorescent microscopes.

In a second aspect, provided herein is a method of spatially labeling cells in a tissue sample, comprising contacting a tissue sample comprising a plurality of cells with the substrates disclosed herein for a time sufficient for at least a portion of the barcode labels and/or reference labels to transfer into at least a portion of the plurality of cells.

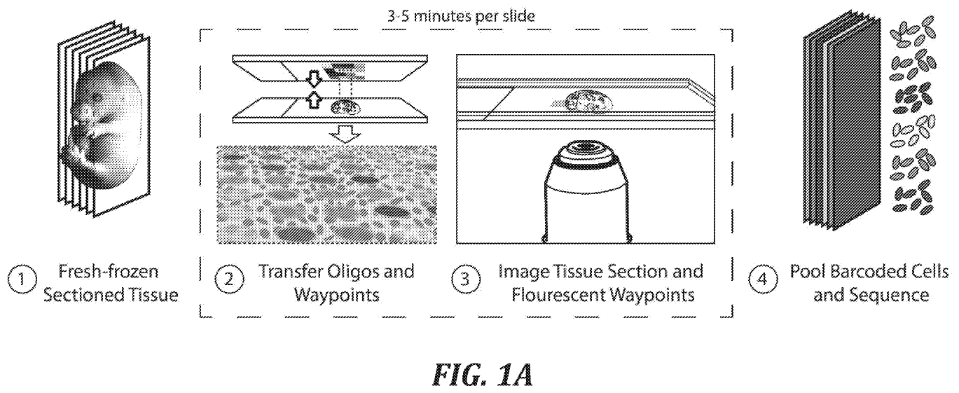

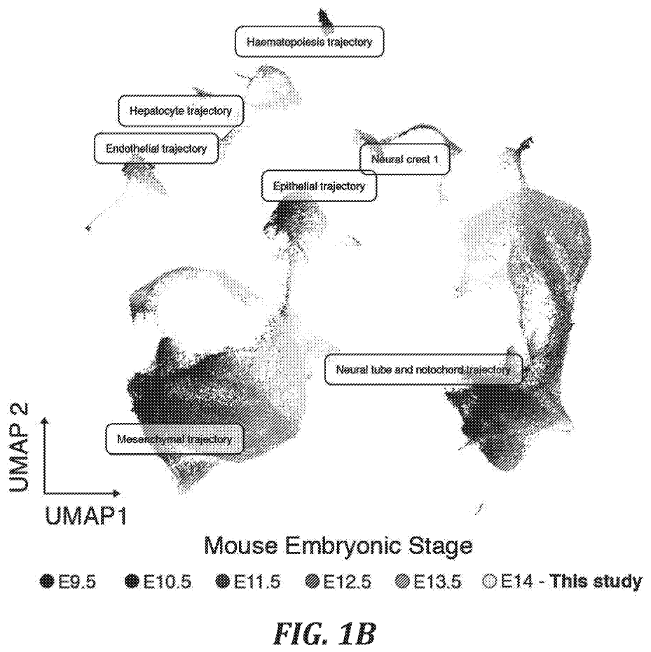

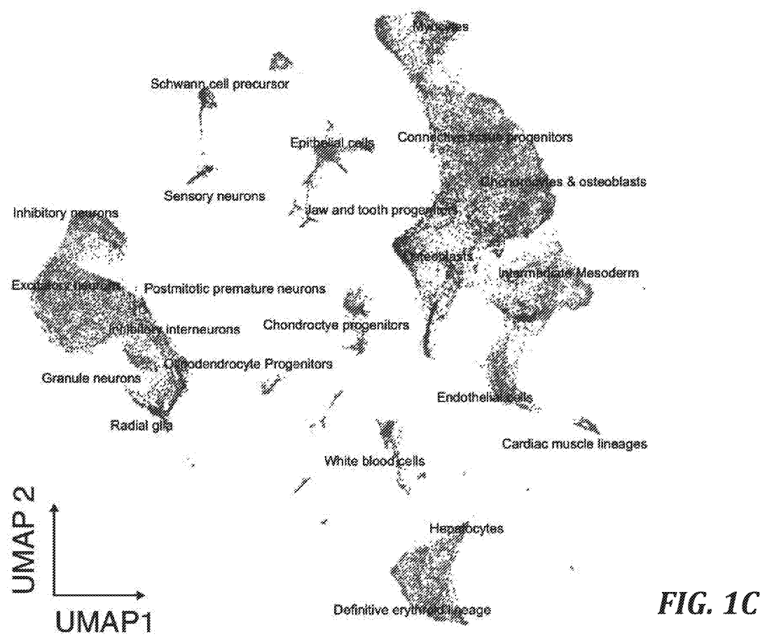

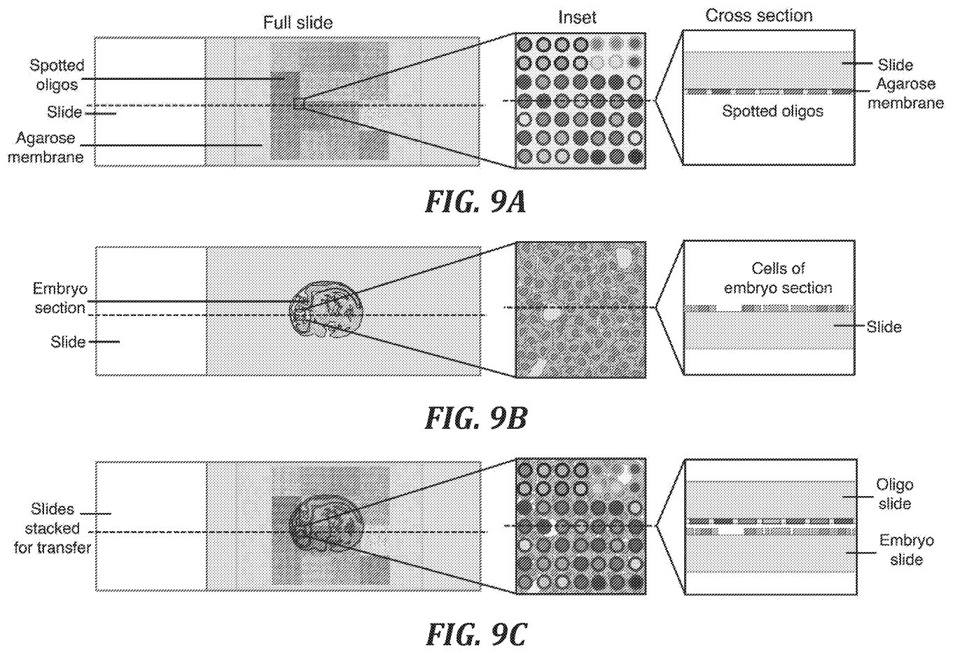

FIGS. 1A, 9, and 10 depict the sequence of the steps of exemplary methods of the disclosure using a substrate described herein.

Any tissue sample can be used in the methods disclosed herein. As used herein, a tissue sample is a sample comprising a plurality of cells, such as tissue slice, tissue biopsy, or a sample taken from a 2D or a 3D tissue culture. The tissue sample can comprise one or more cell types; preferably, the tissue sample comprises two or more cell types. The tissue sample can originate from any biological sample, such as animal sample, human sample, or artificially grown organ or tissue. In some embodiments, the tissue sample is a tissue slice. In some embodiments, the tissue sample is a tissue biopsy. Fresh, frozen, or paraffin-embedded tissue can be used in the methods disclosed herein. In some embodiments, the tissue sample is a tissue slice obtained from flash-frozen fresh tissue sample. In some embodiments, the tissue sample is sandwiched between a glass slide or plastic slide and the substrate during the contacting and the barcode label transfer.

In some embodiments, the time sufficient for at least a portion of the one or more barcode labels to transfer into at least a portion of the plurality of cells is about 1 minute, about 2 minutes, about 3 minutes, about 4 minutes, about 5 minutes, from about 1 minute to about 10 minutes, from about 1 minute to about 5 minutes, or from about 3 minutes to about 5 minutes.

In some embodiments, contacting the tissue sample with a substrate of the disclosure is done in a solution, such as a buffer, that can facilitate the transfer of at least a portion of the one or more barcode labels into the cells, i.e., into the nuclei of the cells, of the tissue sample. In some embodiments, the solution can comprise a cell or tissue permeabilizing agent. In some embodiments, the tissue sample can be permeabilized by pre-treating the tissue sample with a suitable solution comprising a permeabilizing agent prior to contacting with the substrate. Any suitable agent can be used to facilitate the transfer of the labels into the cells of the tissue sample, for example, by diffusion. Examples of cell permeabilizing agents include detergents, such as Triton X 100 and polysorbates, e.g., Tween® reagents. Protocols for cell permeabilization and preparation of permeabilizing buffers are known in the art, for example, those described in Cao J. et al, Comprehensive single-cell transcriptional profiling of a multicellular organism. Science (2017), 357: 6352, pp. 661-667, the disclosure of which is incorporated herein by reference. In some embodiments, the tissue sample is pre-treated with a permeabilizing agent prior to or during contacting the tissue ample with a substrate of the disclosure.

In some embodiments, the solution can comprise one or more compounds or hashes that can uniquely identify the particular substrate used in the method.

In some embodiments, the methods further comprises dissociating the tissue sample into individual cells after contacting the sample with the substrate (e.g., after the completion of the transfer of the barcode labels). In some embodiments, the methods comprise the step of dissociating the tissue sample into individual components, e.g., organelles or cells, after contacting the sample with the substrate. In some embodiments, the dissociated components of the sample can be analyzed separately in parallel and the data from those analyses can be mapped back to the tissue's initial architecture.

For example, in some embodiments, the methods can comprise the following steps: sandwiching a tissue slice comprising a plurality of cells (e.g., permeabilized tissue slice) with a substrate of the disclosure, e.g., a spatially gridded substrate comprising a glass slide support; allowing the tissue slice to remain in contact with the substrate for a time sufficient for at least a portion of the barcode labels to be transferred into the cells; imaging the spatial grid during oligo transfer; extracting nuclei from the tissue; further hashing or labeling with a substrate-specific oligo; chemically fixing; and analyzing gene expression in the tissue.

In another aspect, the disclosure provides a method of spatial gene expression profiling in a multi-cellular sample, comprising:

(a) contacting a tissue sample comprising a plurality of cells with a substrate of disclosed herein for a time sufficient for at least a portion of the one or more barcode labels to transfer into at least a portion of the plurality of cells;

(b) optionally imaging the substrate to determine positions of the reference regions;

(c) extracting nuclei from the cells; and

(d) analyzing gene expression in each individual cell.

In some embodiments, the method comprises dissociating the tissue sample into individual cells after imaging the substrate.

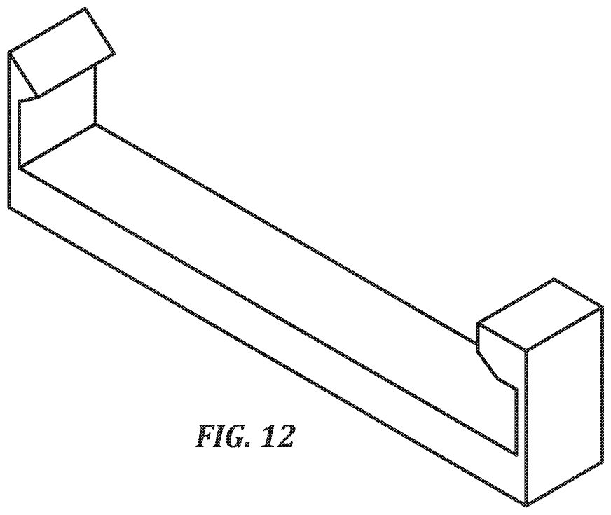

In another aspect, the disclosure provides a kit for spatially labeling cells in a multi-cellular sample, comprising one or more substrates disclosed herein. The kits of the disclosure can comprise one or more additional components, including but not limited to a glass slide (such as a glass slide used to contact the tissue sample with the substrate), permeabilizing buffers, and instructions for use. In some embodiments, the kit of the disclosure further comprises means for securing a multi-cellular sample to the substrate. In some embodiments, the kits comprise a means for allowing to securely sandwich a tissue sample between the substrate of the disclosure and a glass slide, such a holder or a clip. One such exemplary clip is depicted in FIG. 12. The clips can be prepared from any suitable materials, such as natural or synthetic polymers, including but not limited to polypropylene, polyethylene, PTFE, and the like.

While each of the elements of the present disclosure is described herein as containing multiple embodiments, it should be understood that, unless indicated otherwise, each of the embodiments of a given element of the present invention is capable of being used with each of the embodiments of the other elements of the present invention and each such use is intended to form a distinct embodiment of the present invention.

The referenced patents, patent applications, and scientific literature referred to herein are hereby incorporated by reference in their entirety as if each individual publication, patent or patent application were specifically and individually indicated to be incorporated by reference.

As used herein, the term “about” includes plus or minus 10% of the stated value.

While illustrative embodiments have been illustrated and described, it will be appreciated that various changes can be made therein without departing from the spirit and scope of the invention.

As can be appreciated from the disclosure above, the present invention has a wide variety of applications. The invention is further illustrated by the following examples, which are only illustrative and are not intended to limit the definition and scope of the invention in any way.

### Examples

Spatial patterns of gene expression span many scales and are shaped by both local (e.g. cell-cell interactions) and global (e.g. axis, tissue) contexts. However, most in situ methods for profiling gene expression either lack single cell resolution or are restricted to limited fields of view. The substrates and methods of the disclosure, referred to herein as a “sci-Space,” provide a scale-flexible method for spatial transcriptomics that retains single cell resolution while simultaneously capturing heterogeneity at larger scales. In the examples provided below, the sci-Space was applied to the developing mouse embryo, capturing the approximate spatial coordinates of profiled cells from whole embryo serial sections. Genes, including Hox-family and other transcription factors expressed in an anatomically patterned manner across excitatory neurons and other cell types, were identified. It was also shown that that sci-Space can resolve the differential contribution of cell types to signaling molecules exhibiting spatially heterogeneous expression. A new statistical approach for quantifying the contribution of spatial context to variation in gene expression within cell types was developed and applied herein.

Methods for molecular profiling at single cell resolution have the potential to transform the understanding of developmental biology. For example, in diverse models of embryogenesis, the inventors and others have performed “whole organism” profiling of transcription or chromatin accessibility at single cell resolution. Although nearly all major cell types have previously been described, such studies yield a much richer view of their molecular states during development than was previously available. Furthermore, applying machine learning techniques to organize cells in terms of their progression through development has revealed complex branching trajectories similar to, but topologically distinct from, cell lineages.

The spatial organization of cells is central to normal development and homeostasis, as well as to diverse disease processes. However, a key limitation of widely used methods for single cell molecular profiling is that they operate on disaggregated cells or nuclei. Although several groups have developed powerful in situ methods for measuring the expression of many or all genes while retaining spatial information, these methods are not without limitations. A first class of methods, including spatial transcriptomics and SLIDE-seq, count mRNAs at each spot across patterned arrays. However, although they can potentially be implemented at a range of spatial scales, such methods yield aggregate profiles of small regions, rather than resolving individual cells. A second class of methods, including MERFISH, seqFISH, and FISSEQ, measure the expression of many genes while retaining single cell (or even subcellular) resolution within a field of view. However, such multiplex in situ methods are limited by long image acquisition times and complex instrumentation requirements. As such, the tradeoff of high resolution is that assaying whole transcriptomes over large regions becomes impractical.

The present disclosure provides substrates and a spatial transcriptomics method that retains both single cell resolution and the flexibility to acquire positional information at broader scales, e.g. to detect spatial patterns of cell type-specific gene expression that would be found at the level of the embryo, but missed by high-resolution analysis of a small region. Without wishing to be bound by a particular theory, the inventors hypothesized that cells could be labeled with molecular tags that encoded their approximate spatial coordinates within entire tissue sections, and this information can be subsequently recovered without sacrificing single cell resolution. Furthermore, recovering a sample of single cells from across a tissue—in effect a spatially resolved “census” of cells—could reveal spatial patterns of gene expression without comprehensively (and expensively) sequencing the whole transcriptome of every cell.

The so-called sci-Plex, a method for labeling or “hashing” nuclei using unmodified DNA oligos during single-cell RNA-seq with combinatorial indexing (sci-RNA-seq) has been previously developed (Srivatsan, S. R. et al. Massively multiplex chemical transcriptomics at single-cell resolution. Science 367, 45-51 (2020)), which enables the pooling of nuclei from many different specimens or samples into one sci-RNA-seq experiment with minimal marginal cost. To leverage parts of this workflow to capture) spatial information, the inventors first printed unique combinations of barcode labels such as hashing oligos as a spatially defined array and then transferred those oligos to the nuclei of a tissue slice by diffusion. Provided that these hashing oligos were recovered in association with single cell RNA-seq profiles, they could be used to reconstruct each cell's original tissue coordinates upon sequencing.

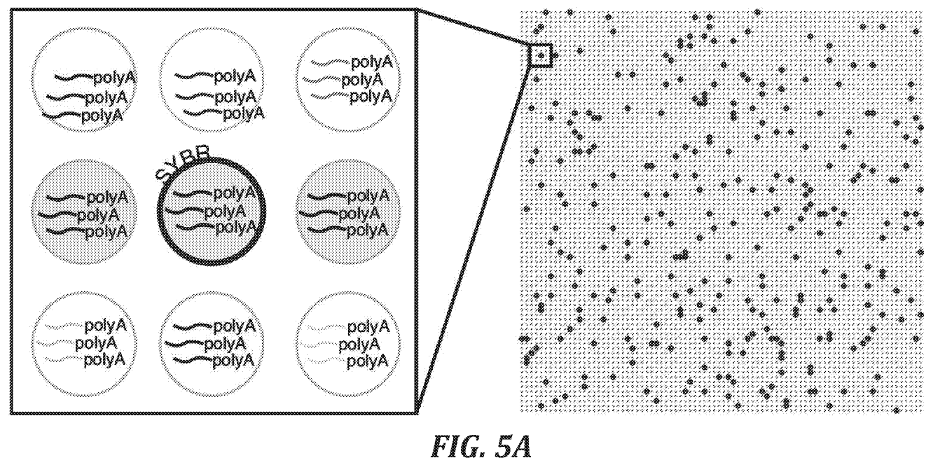

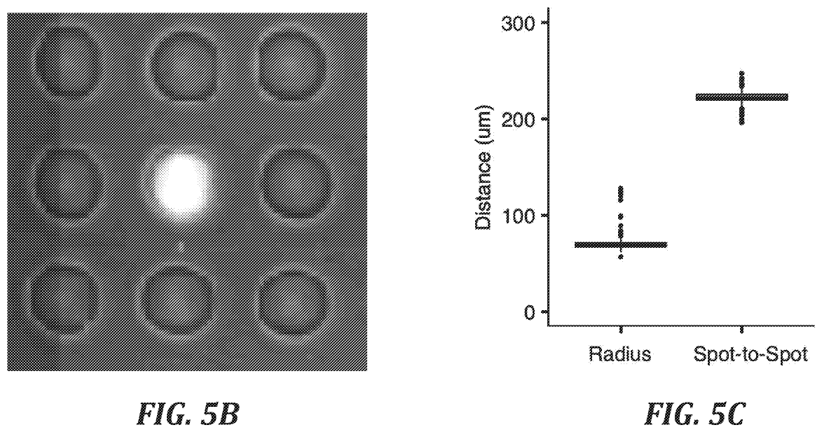

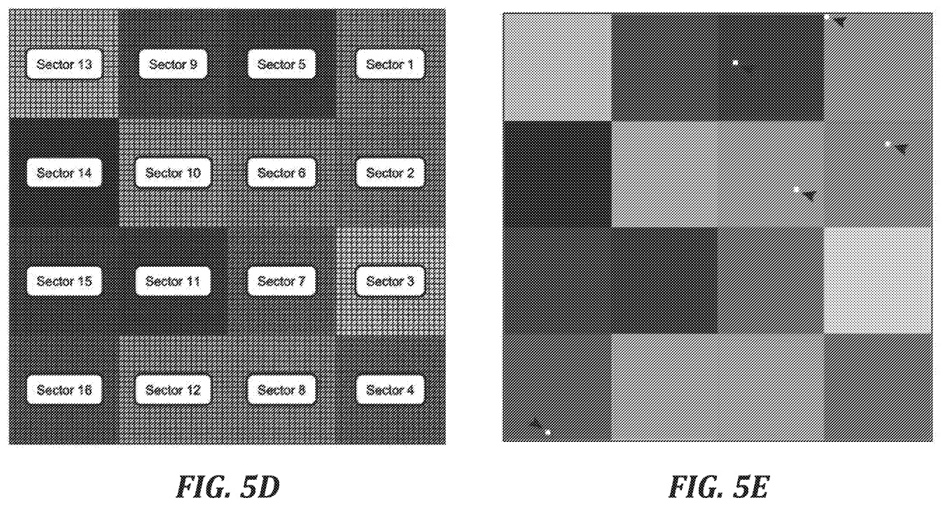

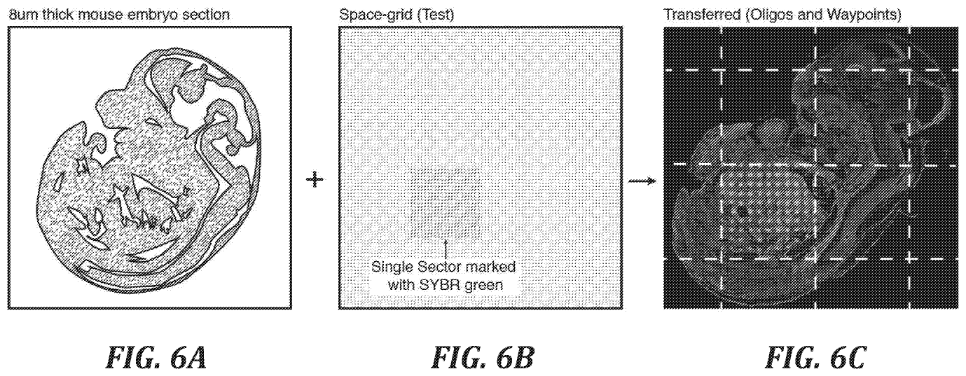

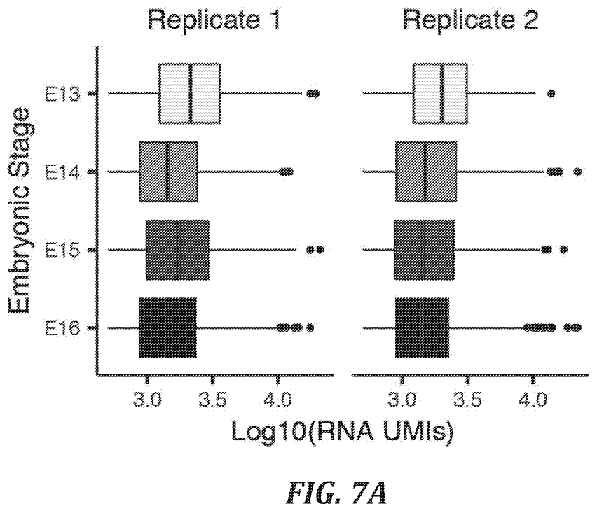

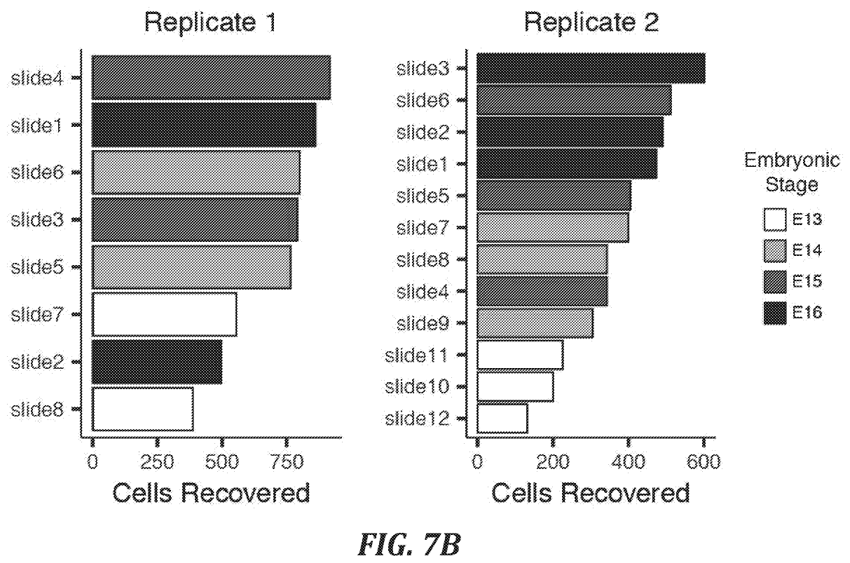

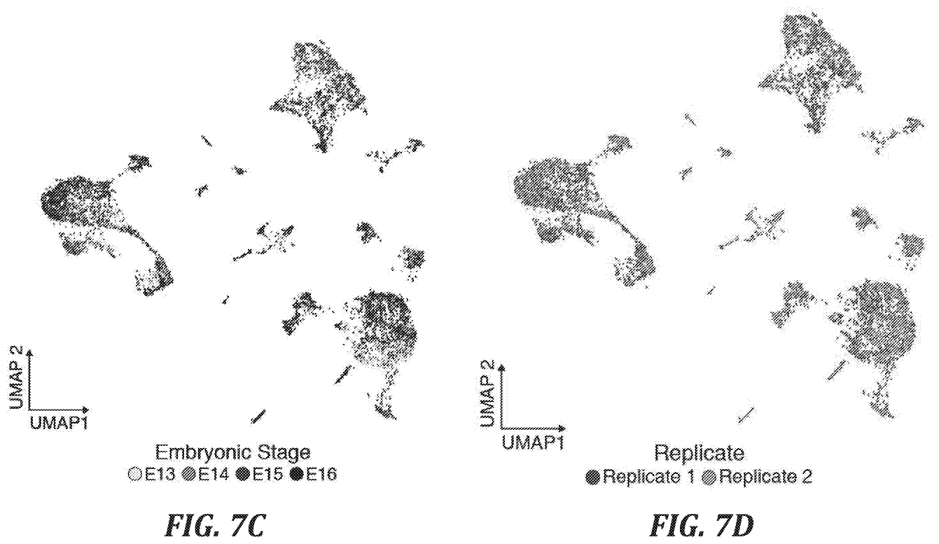

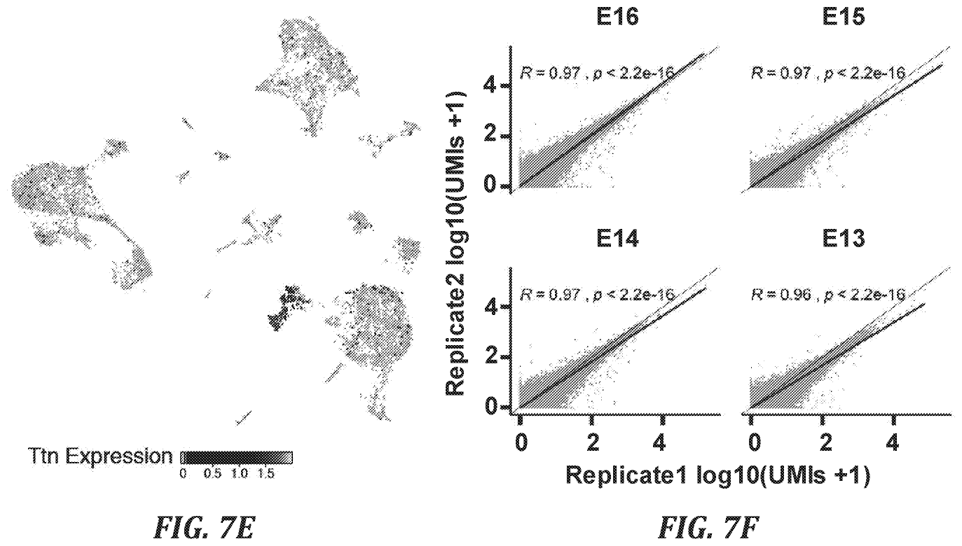

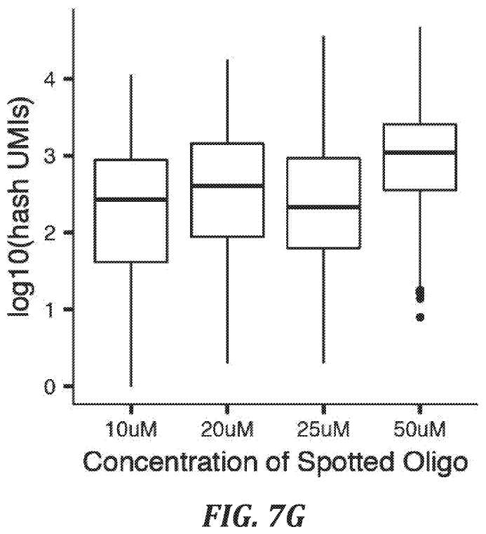

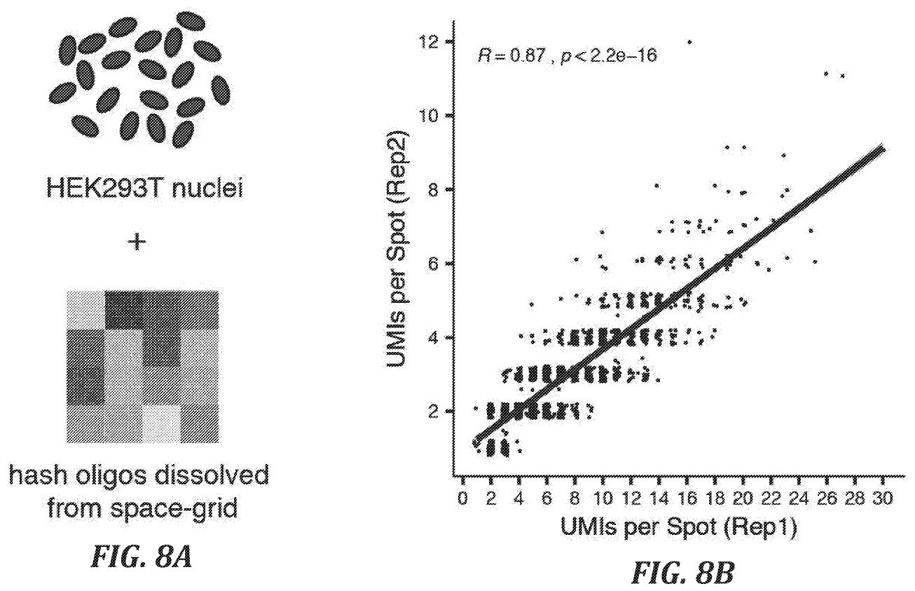

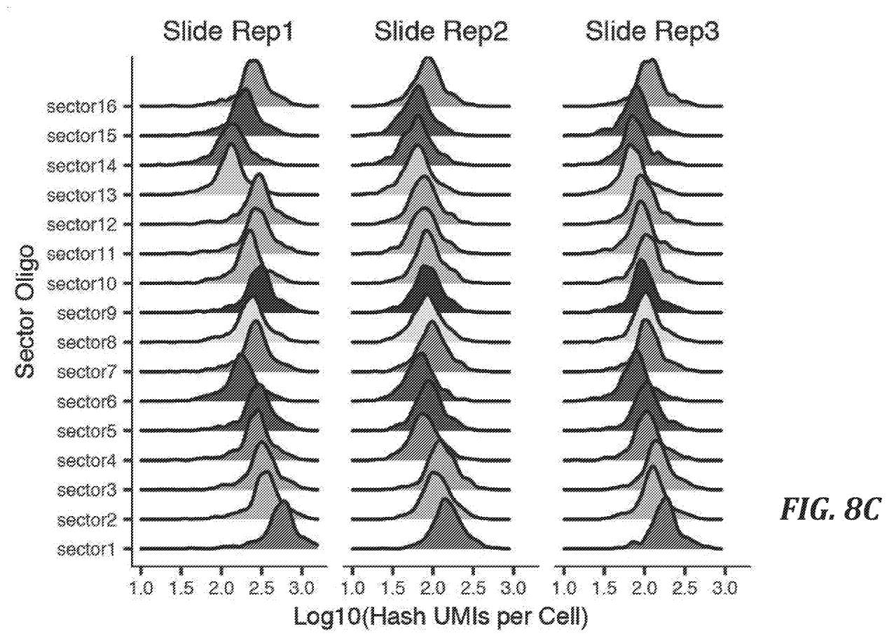

In one exemplary embodiment of the substrate and method of the disclosure (referred to herein as Sci-Space), barcode labels, e.g., hashing oligos, were spotted onto glass slides coated with a thin layer of dried agarose in a gridded format. These spatial grids contained 7,056 uniquely barcoded spots in a 10 mm by 10 mm grid with a mean radius of 73.2±14.1 μm and a mean spot-to-spot center distance of 222±7.5 μm (FIG. 5). About 5% of spots, making up an identifiable pattern, were also loaded with SYBR green fluorescent dye. After transferring the oligos from the slide to the tissue, the grid could be registered with an image of the tissue using these concurrently imaged fluorescent reference points (FIGS. 5 and 6). The hash oligo concentrations can be optimized to robustly label nuclei from sectioned tissue. The inventors developed a protocol for blotting the hash-oligos onto the tissue and verified that single cell transcriptomes from labeled nuclei could be recovered and sequenced (FIGS. 7 and 8). The full exemplary sci-Space protocol of the disclosure consists of four steps: 1) fresh-frozen tissue is sectioned; 2) sectioned tissue is permeabilized and sandwiched with a substrate of the disclosure, e.g., spatially gridded glass slide; 3) during oligo transfer, the spatial grid is imaged; and 4) nuclei from each slide are extracted, further hashed with a slide-specific oligo, chemically fixed and subjected to sci-RNA-seq (FIGS. 1A, 9, and 10).

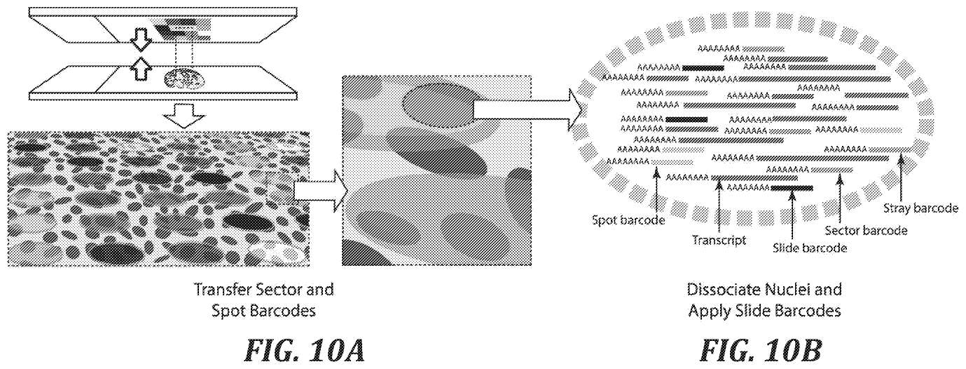

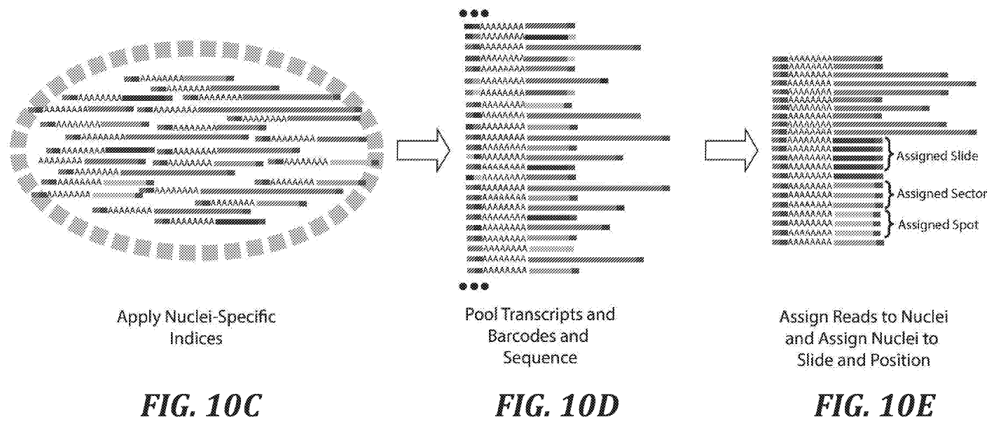

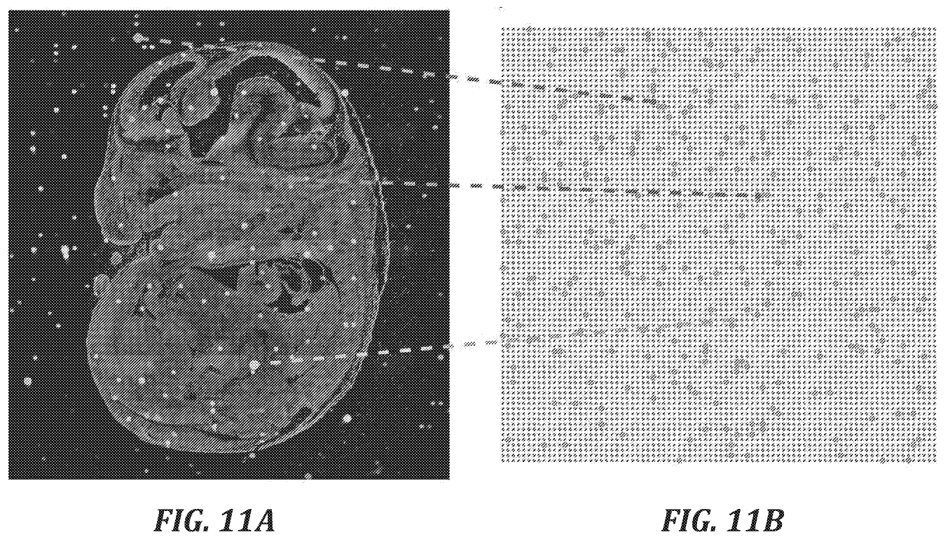

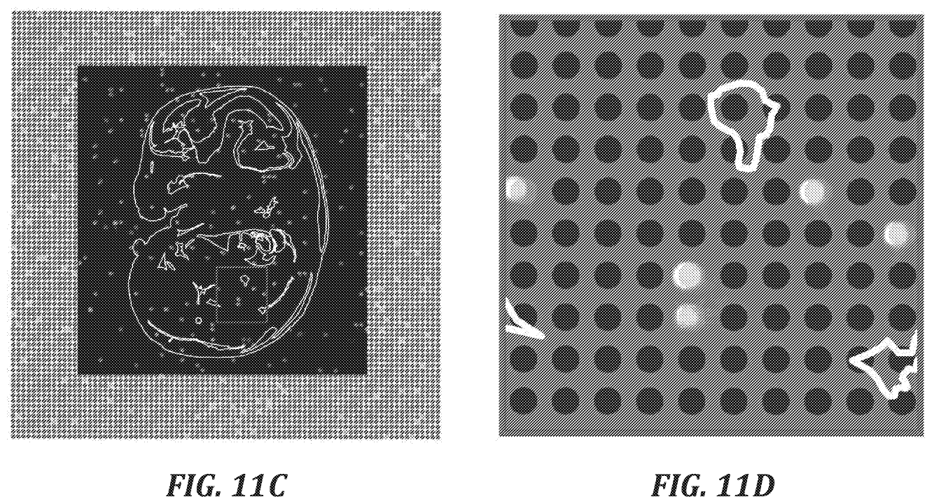

sci-Space was applied to profile six sagittal sections from two different E14.0 C57BL/6N mouse embryos. Based on estimated counts of DAPI stained nuclei obtained by automated analysis of the images, approximately 2.4 million nuclei were present across the six sections (FIG. 10). After losses during buffer exchange and fluorescence activated cell sorting (FACS), obtained 37,689 sci-RNA-seq transcriptomes via two Illumina Nextseq 500 sequencing runs were obtained. After conservative doublet removal and assignment of each cell to a spatial position (based on its slide-, sector- and spot-specific hash oligos), the dataset comprised 26,444 spatially resolved single cell transcriptomes with a median of 2,400 unique molecular identifiers (UMIs) and 1,260 genes detected per cell, and without apparent batch effects between slides. Rather than annotating cell types ab initio, these data were co-embedded with published sci-RNA-seq “mouse organogenesis cell atlas” (MOCA) data spanning E9.5 to E13.5. Reassuringly, these E14.0 data distributed to the distal tips of various E9.5 to E13.5 trajectories (FIG. 1B). Cell types were then inferred via nearest neighbor label transfer from adjacent E13.5 cells (FIG. 1B). The cell type labels obtained by these procedures were highly concordant with those obtained with Garnett, a semi-supervised method for annotating single cell data.

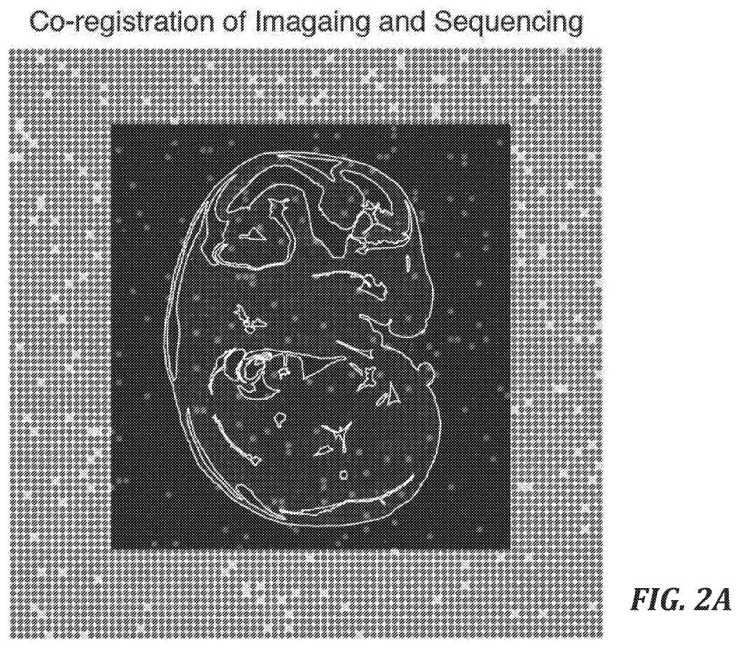

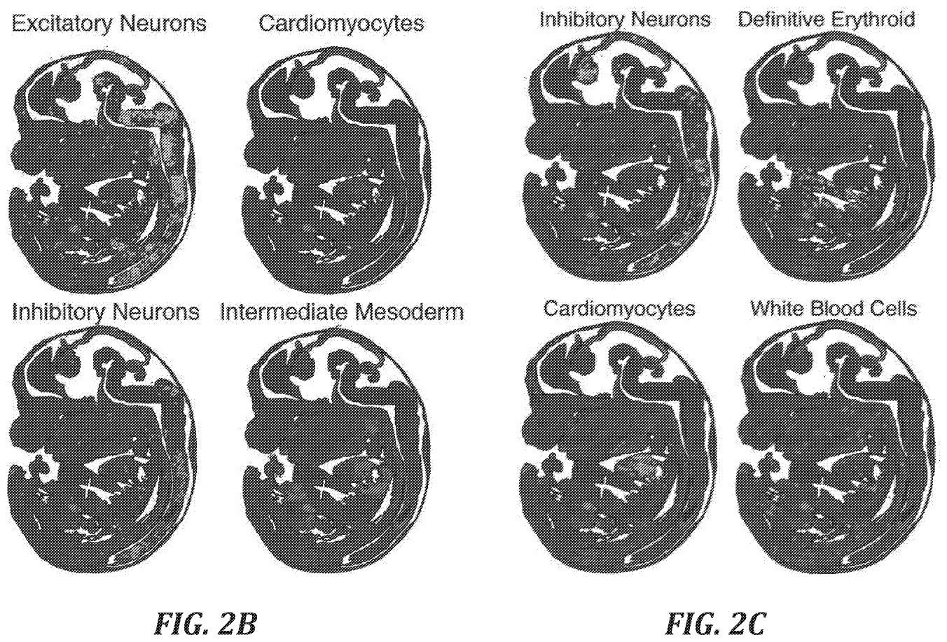

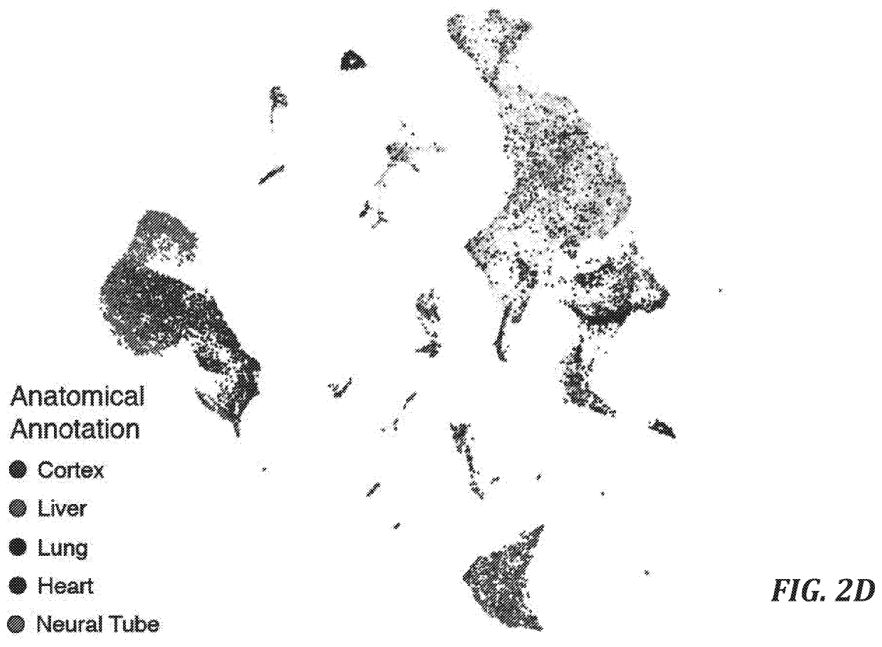

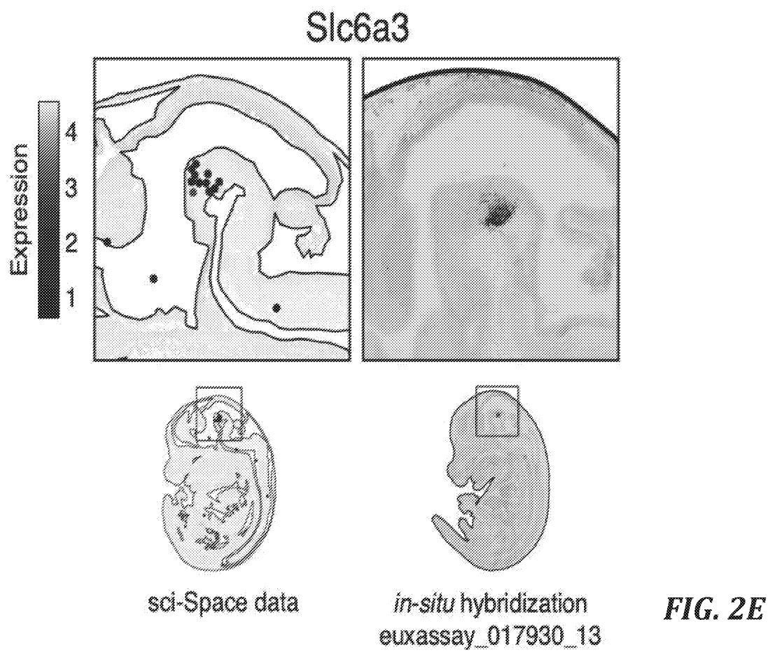

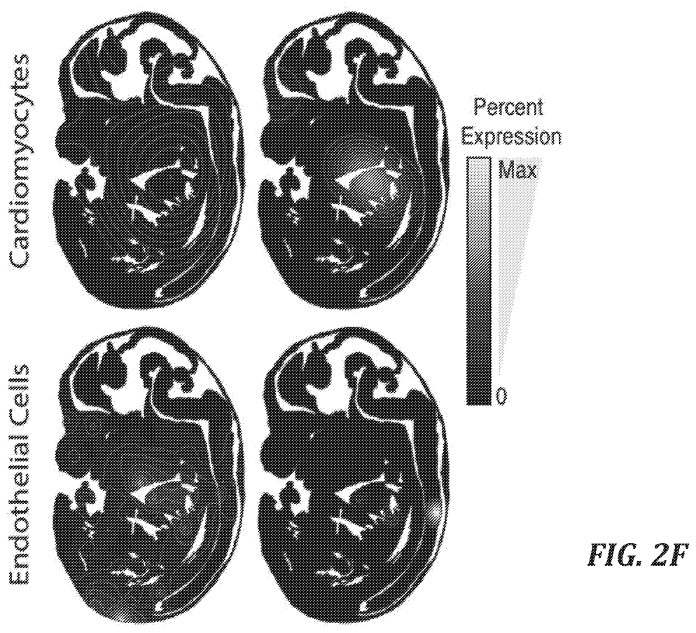

Images of sectioned embryos and sequencing data were co-registered by matching the SYBR waypoints in the oligo grid to identifiable spots of SYBR staining in the image. (FIG. 2A). This alignment yielded an affine transformation from grid positions to the image, from which cell coordinates were then calculated within the sectioned tissue.

As an initial assessment of spatial mapping accuracy, the distribution of cell type annotations within each section can be visualized. To facilitate this, each slide's image was segmented into readily discernible organs (FIG. 2B), a process aided by immunostaining and alignment of adjacent sections. As expected, neuronal cells mapped largely within the spinal cord and cortex, cardiomyocytes to the heart, and white blood cells throughout the organism (FIG. 2C). Although several cell types mapped almost exclusively to one anatomic segment (e.g. hepatocytes to liver), others mapped more broadly (e.g. differentiating mesenchymal and endothelial cells) (FIG. 2D). Finally, wherever cells were captured, sci-Space enabled the visualization of any gene in the transcriptome akin to a digital in situ hybridization. For example, a sci-Space-based digital in situ shows a cluster of excitatory neurons expressing the dopamine transporter Slc6a3 at the midbrain-hindbrain boundary, consistent with stage- and section matched whole-mount in situs (FIG. 2E).

Current spatial transcriptomics methods relying on patterned arrays aggregate all transcripts observed within small regions comprising many cells. Such aggregation compromises the ability to discern the transcriptional state of single cells. To illustrate this point using sci-Space data, 3,602 spatial positions that contained two or more cells were aggregated. Dimensionality reduction of aggregated spatial positions demonstrated that cell subtypes and rare cell types were not clearly separated in comparison to downsampled single cell data. As such, the aggregate approach risks missing or confounding the source of spatial heterogeneity. For example, in the heart, cardiomyocytes appear to be the sole source of Myh6 expression, whereas the growth factor Igf2, although also spatially restricted, is produced by endothelial cells (FIG. 2F).

To systematically examine these data for spatially patterned, cell type-specific gene expression across the whole embryo, spatial autocorrelation was quantified—the tendency of spatially proximal cells to express similar levels of a given gene. Testing for spatially autocorrelated genes within each annotated cell type revealed hundreds to thousands of genes per cell type per slide with positive spatial autocorrelation (FIG. 3A; Moran's test, FDR<0.001, FIG. 5). The differentiating mesenchyme and excitatory neurons had the most genes detected by this analysis (mean 4,334 and 3,467 spatially autocorrelated genes per slide, respectively). Importantly, such spatial autocorrelation could result from the presence of spatially restricted, unannotated cell subtypes or states, or from spatially restricted gene expression that is not tied to cell subtype or state. For example, sub-clustering of differentiating mesenchyme revealed multiple spatially restricted cell subtypes.

To better distinguish between spatially restricted genes vs. subtype/state restricted genes, the inventors compared each gene's spatial autocorrelation to its autocorrelation in UMAP space, an indicator of cell subtype/state restricted genes. The analysis was focused on excitatory neurons, a cell type wherein subtypes were less apparent upon sub-clustering. Three classes of genes were observed within each tissue section (FIG. 3B): 1) genes with spatially restricted expression that do not appear restricted by cell subtype/state; 2) genes with correlated spatial and subtype/state restriction; 3) genes with restricted subtype/state expression that do not appear spatially restricted.

Amongst the genes restricted by excitatory neuron subtype/state were the transcription factors Gli2 (FIG. 3C) and En1 (FIG. 3D). Gli2 specifies the patterning of neurons in the ventral spinal cord, and En1 is expressed by cells in the isthmic organizer, a structure which directs neural differentiation at the midbrain-hindbrain boundary. The sci-Space data show that cells which express these genes were either spatially scattered, in the case of Gli2 (FIG. 3C) or spatially restricted, in the case of En1 (FIG. 3D).

The inventors were particularly interested in those genes that exhibited clear spatial restriction but without restriction by excitatory neuron subtype or state. Amongst these genes was Cyp26b1, an enzyme that metabolizes the developmental morphogen retinoic acid, a key driver of neuronal differentiation along the anterior-posterior (AP) axis of the spinal cord (FIG. 3E). The sci-Space data reveal localized expression of Cyp26b1 predominantly in the brainstem, a region previously shown to catabolize retinoic acid. Other genes exhibiting punctate expression unrestricted by excitatory neuron subcluster, such as L3mbt11, Calb1, and Col9a1, spanned diverse functions previously uncharacterized in development (FIGS. 3B and 3F).

Notably, this subset of genes was also enriched for Hox genes, a class of homeotic transcription factors which specify the body plan (FIGS. 3G-I). Genes in the HoxC cluster were expressed in a manner reminiscent of the establishment of an AP axis of the spinal cord in both excitatory (FIG. 3I) and inhibitory neurons and were not strongly autocorrelated in UMAP space. To confirm this result was not an artifact of cellular sampling or sequencing depth, the same set of genes in a single cell RNA-seq atlas of the developing mouse spinal cord was visualized; in these data, genes in the HoxC cluster are also unrestricted by excitatory neuron subtype or state.

To discover additional genes exhibiting AP patterning akin to the Hox genes, a differential gene expression test using each cell's position along the AP axis of the spinal cord was performed. This analysis detected 150 genes, including 35 transcription factors, many with established roles in development such as Sox2, Olig1, and Hopx (FDR<0.05; FIG. 3J). Several of these factors regulate neuronal maturation (e.g. Nfia, Nfib, Tcf4 and Zic1), suggesting that at least some of the observed AP patterning might correspond to heterogeneity in the developmental maturity of cells along the spinal cord.

Asynchrony in neuronal maturation is well-documented along the dorsal-ventral axis (DV), while maturation along the AP axis is generally thought to proceed in an anterior-to-posterior manner. To assess the relationship between AP patterning and neuronal maturation in the data, sci-Space cells from this region were aligned to the aforementioned spinal cord atlas. The joint embedding identified a transcriptional trajectory, corresponding to neuronal development, along which sci-Space cells were interspersed (FIGS. 3K-L). Although progenitors and more mature neurons were intermingled along the entire AP axis, two foci of progenitors were observed, one near the brain-spinal cord junction and second in the mid-posterior region (FIG. 3L). Taken together, these analyses suggest a more complex relationship between AP position and the timing of neuronal maturation than a simple anterior-to-posterior gradient. More generally, they also suggest that a substantial proportion of spatially patterned expression may correspond to asynchronous differentiation.

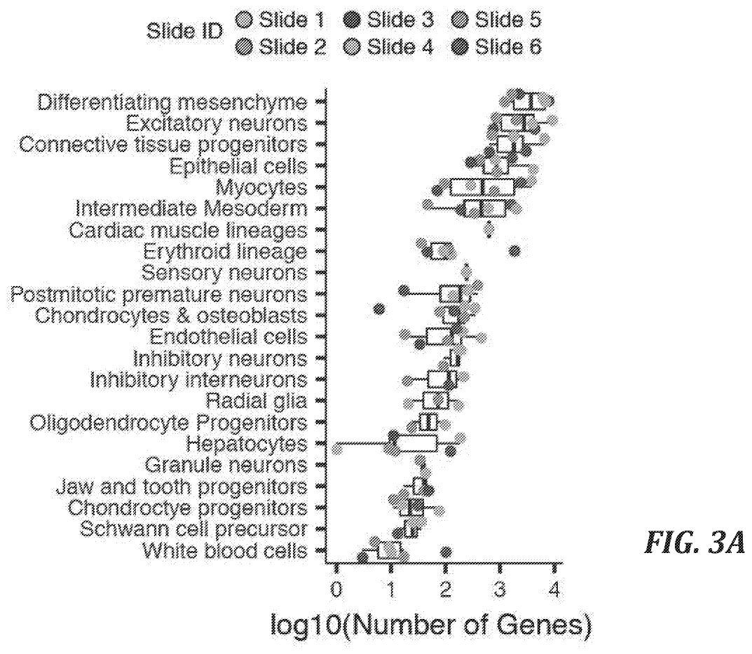

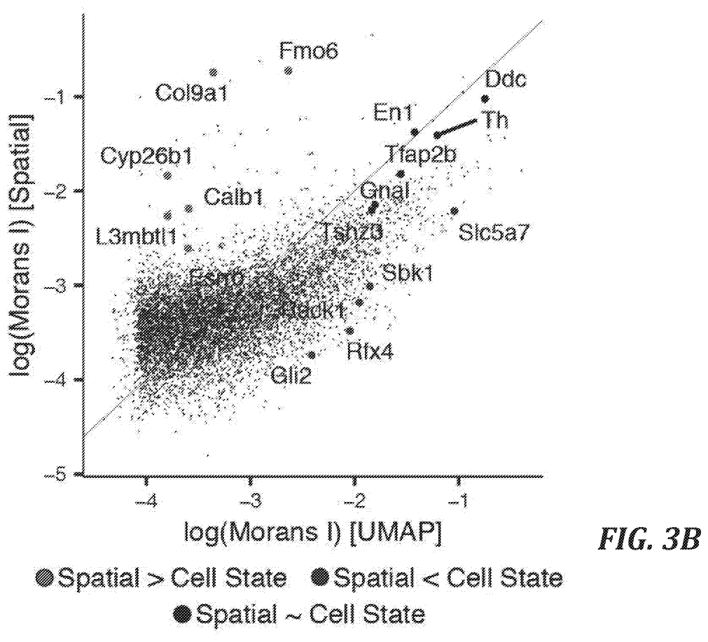

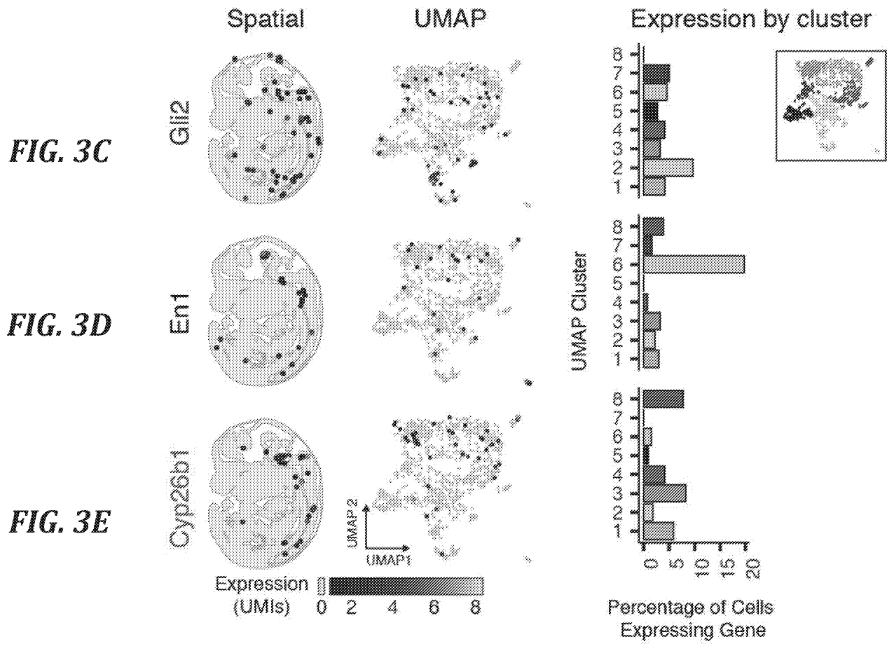

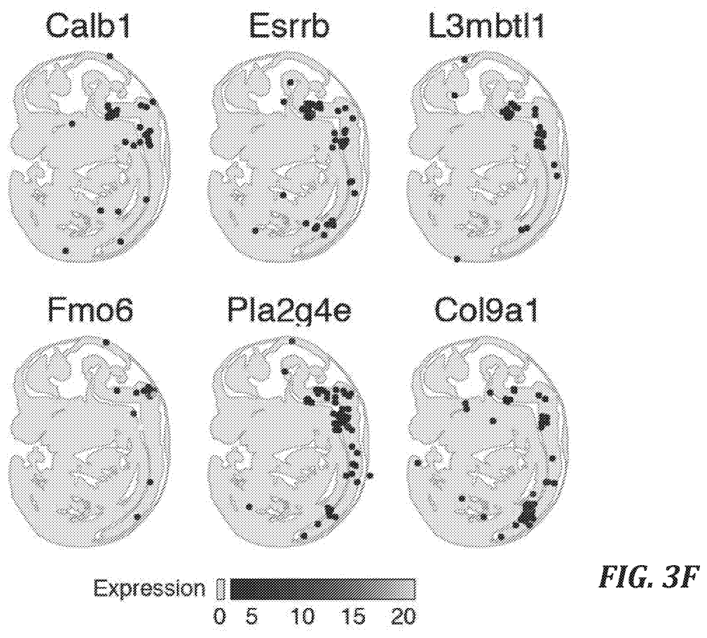

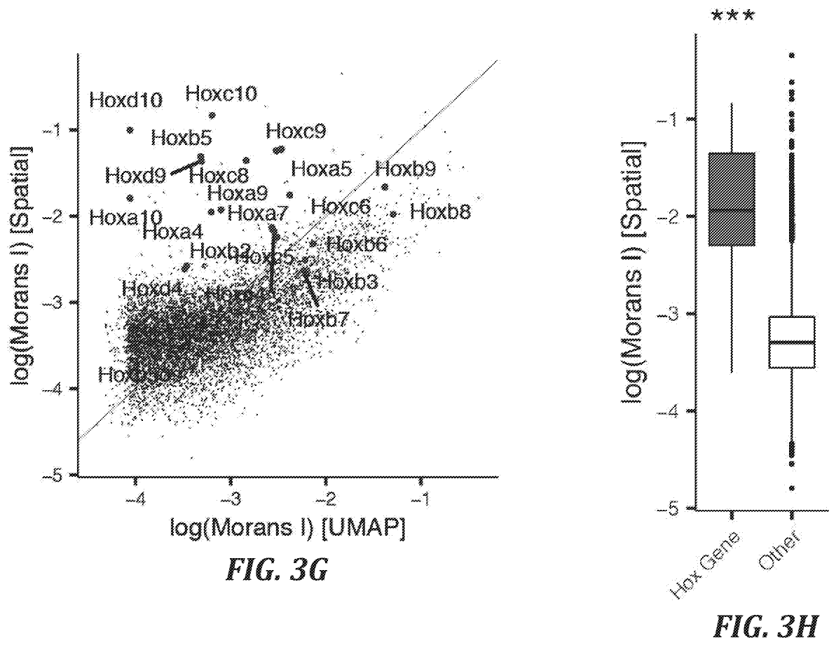

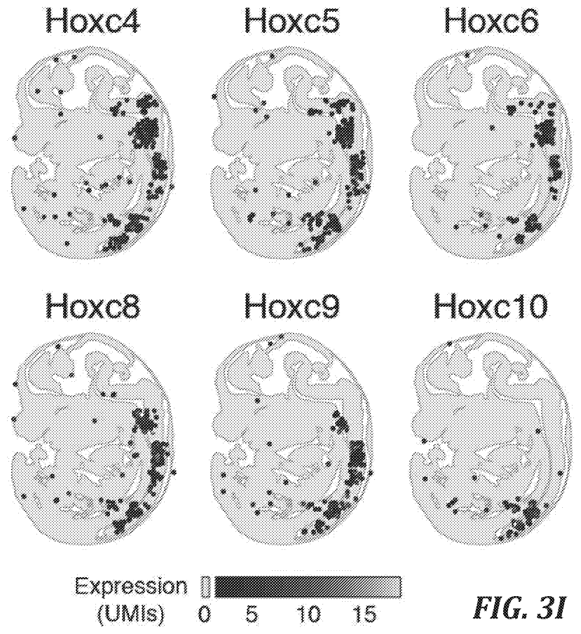

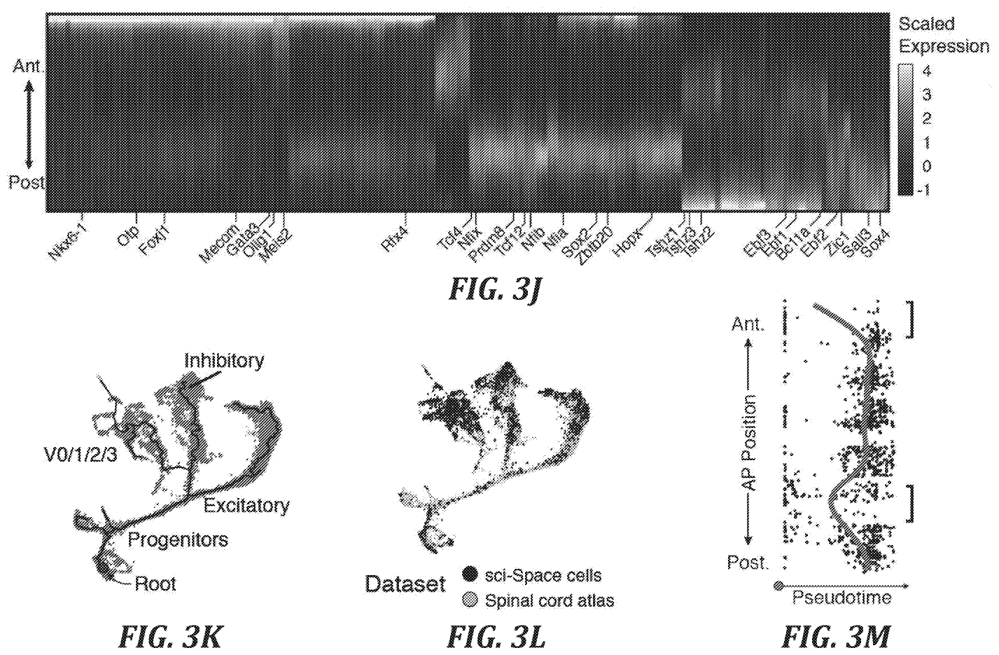

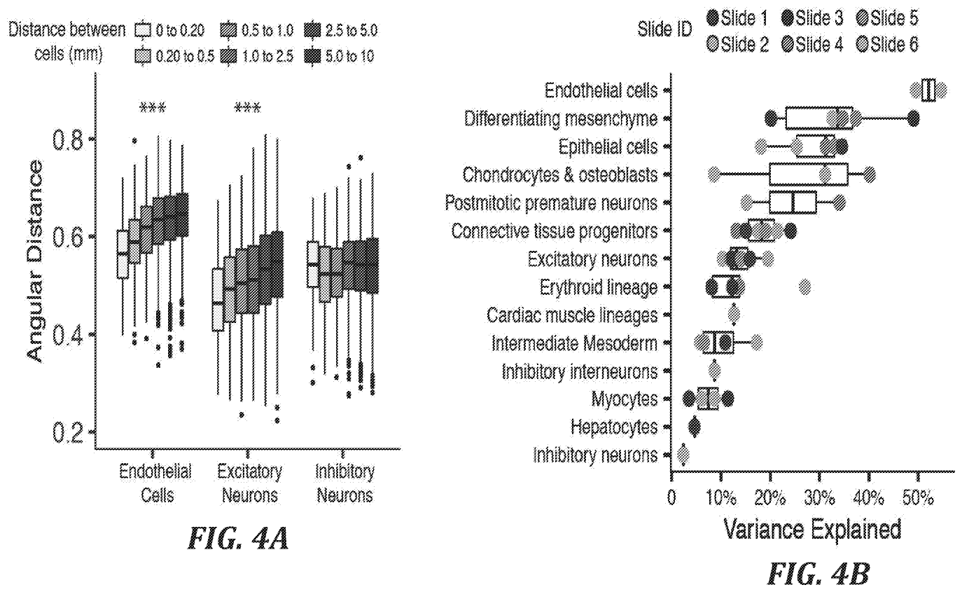

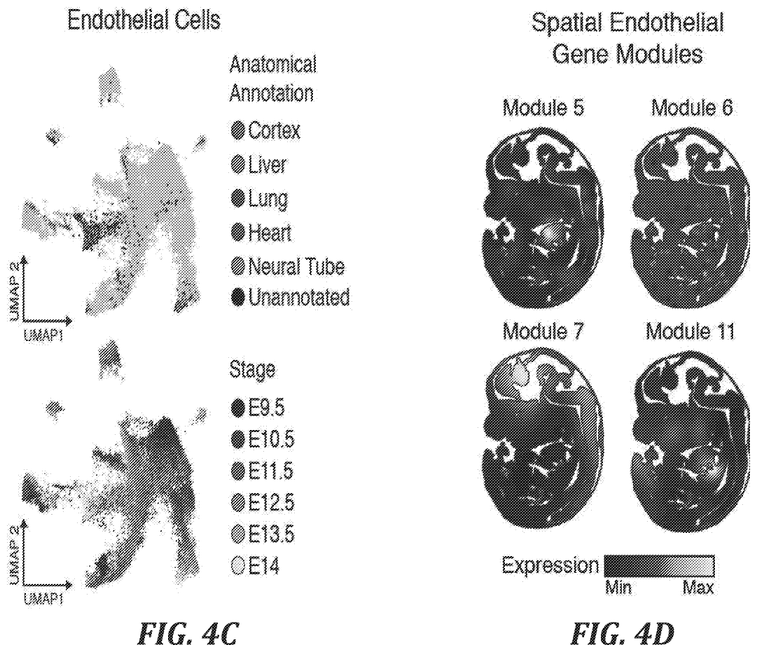

Next, rather than examining individual genes, the inventors sought to understand how each cell type's transcriptome spatially varied across the embryo. The angular distance between the global transcriptomes of pairs of cells of the same type located at varying distances from one another was calculated. For many cell types, as the physical distance between cells increased, so did the angular distance between their transcriptomes. However, this trend varied by cell type, e.g. it was particularly pronounced in excitatory neurons and endothelial cells, but undetectable in inhibitory neurons (FIG. 4A).

A new statistical approach for quantifying the contribution of spatial context to variation in global gene expression across individual cells was next developed. Briefly, the cells were first partitioned into groups based on cell type and spatial location. Then, the angular distance between each cell and the average expression profile for cells of that same type in the same spatial bin was computed. Some variance across cells was technical, due to sampling only a fraction of transcripts in each cell. The inventors estimated the technical variance attributable to data sparsity by simulating single cell UMI count profiles from each of the spot averages. After subtracting technical variance from total variance, the inventors were able to quantify how much of the remaining biological variance was due to each cell's type and spatial position. The variance that one could expect to explain using this approach under a null model that permuted cell type and spatial position labels was also estimated.

Before applying this variance decomposition approach to sci-Space data, the inventors sought to validate it on another dataset for which sources of transcriptional variation across cells are better understood. Packer et al. previously analyzed developing C. elegans embryos, which have a defined, deterministic lineage, and observed that a cell's position in the lineage is highly predictive of its gene expression profile. The inventors therefore decomposed variance across cells from this dataset by grouping cells that shared a common parent in the worm lineage. According to the Law of Total Variance, total variance across cells should equal the variance within these groups plus the variance between group averages. Reassuringly, this was the case. In agreement with Packer et al., the new approach disclosed herein showed that variance attributable to a cell's position in the C. elegans lineage peaked around generation 7.

The variance decomposition approach was then applied to the sci-Space data disclosed herein. Variance attributable to sparse UMI sampling accounted for 93.5% of the observed variance in global gene expression across cells. In subsequent analyses, the inventors focused on the remaining 6.5% “non-sampling” variance and found that spatial position within the mouse embryo contributes to transcriptional heterogeneity within many cell types. Across all cells in the dataset, cell type alone accounted for 23% of the non-sampling variance in global gene expression. Spatial position, after accounting for cell type, explained a further 23%. However, some cell types' transcriptomes appeared far more sensitive to spatial position than others (FIG. 4B). For example, differentiating mesenchyme and chondrocyte progenitors were highly influenced by position within the embryo, possibly reflecting the ongoing development of the connective and skeletal tissues at E14.0. In contrast, the transcriptomes of inhibitory neurons did not appear to vary much across the embryo, consistent with a previous report that cortical inhibitory neurons lack differentially expressed genes between different layers of the adult mouse brain.

Spatial position accounted for 52% of non-sampling variance across endothelial cells, which have been previously reported to specialize into subsets with organ-specific roles in adult mice. With MOCA, the inventors identified several subsets of endothelial cells with expression programs indicative of organ specialization, but because MOCA was constructed without spatially-resolved expression data, the organ-specific annotations were limited and preliminary. To evaluate whether MOCA endothelial cell subtypes were indeed restricted to different developing organs, the inventors aligned and examined endothelial cells from the sci-Space and MOCA datasets. The resulting co-embedding revealed that cells from the two datasets were arranged spatio-temporally, such that cells formed temporal branches corresponding to the development of distinct anatomically restricted endothelial cells (FIG. 4C). MOCA endothelial cells putatively from the brain, liver, and heart co-localized with sci-Space cells from these anatomic segments, confirming the original MOCA annotations. Next, genes were organized into ‘modules’ that shared a common pattern of spatial regulation. Several of these modules were clearly elevated in endothelial cells within specific organs. For example, genes in module 5 were specifically expressed in the heart, while module 7 was restricted to the brain and spinal cord. In contrast, module 6 genes were expressed more uniformly over the embryo but were enriched for functions in arterial endothelial cells (FIG. 4D). Finally, some spatially restricted modules, such as module 11, consisted largely of genes associated with proliferation and catabolism indicative of organ morphogenesis and growth. In line with other reports describing organ-specific endothelial cell subtypes, these observations further demonstrate that spatially-patterned endothelial cell diversity is established during organogenesis.

The sci-Space disclosed herein provides a new method for spatial transcriptomics that retains single cell resolution while capturing spatial information at a scale specified by a patterned array of cell hashing oligos. sci-Space was used herein to retrieve the approximate spatial coordinates of transcriptionally profiled cells across serial sections from E14.0 mouse embryos. Using the method, the inventors identified examples, some expected and others novel, of genes expressed in an anatomically patterned manner within cells of a given type. As highlighted in the spinal cord, components of this spatial patterning are likely attributable to heterogeneous expression of transcription factors as well as ongoing, asynchronous differentiation. The sci-Space data are readily integrated with non-spatial single-cell RNA-seq data previously collected from mouse embryos at adjacent timepoints, enabling rapid annotation of diverse cell types and visualization of cell type-specific, spatially patterned gene expression, i.e. digital in situs.

As shown herein, sci-Space fills a need not addressed by other technologies. Like in situ hybridizations, spatial transcriptomics or SLIDE-seq, sci-Space can be applied efficiently to large regions, e.g. whole embryo serial sections. However, unlike these methods, sci-Space recovers single cell transcriptomes. It can therefore capture patterns of spatial gene regulation private to specific cell types and estimate the contribution of each cell type to the expression of morphogens and other signalling molecules, both within and across anatomical regions.

At the other end of the spectrum, seqFISH, MERFISH, FISSEQ and similar methods can provide single cell resolution and are readily capable of detecting gene expression gradients over short distances. On the other hand, the complex instrumentation and long imaging times that these technologies require are impractical to routinely apply at the scale of entire tissues, organs or embryos. In contrast, sci-Space can be performed on a conventional widefield microscope and imaging takes minutes per slide. sci-Space furthermore captures the global gene expression program and does not require the design and validation of gene-specific probes.

An additional advantage of sci-Space over all contemporary alternative technologies is that because single cells are profiled ex situ, sci-Space can potentially adapted for spatial profiling of any aspect of single cell biology for which a sci-method has been developed (e.g. chromatin accessibility, methylation, protein-DNA binding, etc.), simply by adding steps to co-assay the hashing oligos.

The inventors also developed a statistical approach to identify cell types in the developing embryo with spatially regulated gene expression. This method quantified the contributions of spatial position, cell type, and technical factors to decompose the variance across cells' transcriptomes. It was found that in some cases, spatial context explained as much variance as cell type, with endothelial cells and certain mesenchymal cell types appearing to be particularly associated with spatial position at the resolution examined, and inhibitory neurons particularly ignorant of it.

If contemporary single cell profiling methods are more analogous to a “census” than an “atlas”, sci-Space might be thought of as providing zip codes for each queried cell in a national census. Together with data from complementary technologies, the further application of sci-Space to serial sections spanning entire embryos from many timepoints can facilitate the construction of a highly time- and space-resolved atlas of gene expression during mammalian development.

Preparation of Exemplary Substrates

A thin membrane of dried agarose was fabricated on the surface of microscope slides (Superfrost Plus, Thermofisher). This agarose matrix absorbed and retained an array of spotted oligo hashes. To prepare nuclease-free agarose, 3% w/v low melting temperature agarose powder (SeaPlaque, Lonza, Bend, Oreg.) was added to deionized water containing 0.1% v/v diethyl pyrocarbonate, incubated 2 hr at room temperature, and autoclaved for 15 min. The uniform thickness of the layer of agarose across the slide surface was patterned using spacers of two stacked 22×22 mm, number one thickness (0.15±0.02 mm each) coverslips overhanging either end of the slide. Molding of the agarose was performed by pipetting a 300 uL volume of heated agarose solution into the center of the slide and slowly placing a second slide onto the agarose solution avoiding the formation of bubbles. The molding slide was allowed to rest on the cover glass spacers. After the agarose had gelled between the two slides (˜30-60 min on ice) a razor blade was used to release the exposed edges of the agarose layer from the top, molding slide. The two slides were then carefully slid apart and the cover glass spacers were removed. The resulting thin layer of agarose gel was dried onto the bottom slide overnight in a biosafety cabinet. All agarose slides were UV-treated for 20-30 min prior to spotting to further protect against nuclease activity.

The space-grid array of barcoding labeling regions, e.g., spots containing hashing oligos, and reference regions, e.g., SYBR green reference points, was spotted onto agarose-coated slides using a QArray2 microarray scanner (Genetix, New Milton, Hampshire, GB). A series of 384-well high sample recovery plates (Molecular Devices, San Jose, Calif.) was prepared containing a final concentration of 15 uM spot oligo and 2.5 uM sector oligo per well (Integrated DNA Technologies, Coralville, Iowa), and 0.5% v/v glycerol, with or without SYBR green dye ([5×] Thermofisher) to achieve the predetermined oligo and SYBR green reference point layout when a 21×21 spot/pin array was printed with 16 spotting pins (4×4 grid). These printing parameters gave space-grids containing 7056 (84×84) spots of unique oligo combinations. The spotting height was adjusted to ensure consistent contact of the spotting pins with the transfer slides' agarose coating.

Testing Blotting Concentrations of Hash Oligos and SYBR Green

Space-grids for testing hash oligo blotting concentrations were prepared as noted above using the QArray2 microarray scanner (Genetix, New Milton, Hampshire, GB). Each space-grid was given a single distinct DNA barcode sequence at a chosen final concentration (10 μM, 20 μM, 25 μM, or 50 μM) with a single sector marked with 5×SYBR green. These space-grids were then blotted onto a series of mouse embryo sections ranging from E13 to E16 (Zyagen, San Diego, Calif.). First, permeabilization and hashing solution was prepared for each slide by mixing a unique slide-specific hash oligo (5 μL at 10 μM) in the 495 uL permeabilization solution [10 mM Tris/HCl pH 7.4, 10 mM NaCl, 3 mM MgCl2 with 1% v/v superase inhibitor (Invitrogen) and 0.1% v/v IGEPAL CA-630 (Sigma Aldrich)]. Following permeabilization, each slide was barcoded via transfer with a test space-grid. The transfer was then imaged and the cells were harvested by cell scraping into a solution of 5% paraformaldehyde (cat no. 100504-940, VWR) in 1×PBS. After 15 minutes of fixation on ice, cells were centrifuged (800 g for 10 minutes), pooled and subjected to sci-RNA-seq2 library preparation. These libraries were then sequenced on a Nextseq 500 (Illumina, San Diego, Calif.) using a high output 75 cycle kit (Read 1: 18 cycles, Read 2: 52 cycles, Index 1: 10 cycles and Index 2: 10 cycles).

Testing Spotted Space-Grids

Hash oligos from three space-grids were dissolved in 500 μL of permeabilization solution [10 mM Tris/HCl pH 7.4, 10 mM NaCl, 3 mM MgCl2 with 1% v/v superase inhibitor (Invitrogen) and 0.1% v/v IGEPAL CA-630 (Sigma Aldrich)]. Concurrently, three aliquots of 2 million HEK29T cells were harvested and washed once with 1×PBS. The resuspended hash oligo solutions were then used to lyse and label the HEK293T nuclei. After a 3-minute incubation on ice, the nuclei suspension was chemically fixed with 5 mL of 4% paraformaldehyde and incubated for 15 minutes on ice. Nuclei were then pelleted at 500 g for 5 minutes, washed, with 500 μL Nuclei Buffer (NSB) [10 mM Tris/HCl pH 7.4, 10 mM NaCl, 3 mM MgCl2 with 1% v/v superase inhibitor (Invitrogen) 1% v/v BSA (New England Biolabs)] and permeabilized by resuspension in 500 μL NB+0.2% Triton-X. These nuclei were centrifuged and washed with 500 uL of NSB. These nuclei were then pelleted and 5000 nuclei from each sample was loaded into indexed reverse-transcription reactions. Reverse transcription was performed as described previously; were pooled and 25 nuclei were sorted into a 96 well plate containing 16 μL of elution buffer per well. Libraries were prepared by performing an indexed PCR using 20 uL of NEBNext High-Fidelity 2×PCR Master Mix (NEB), 2 μL of 10 μM indexed P5 primer and 2 μL of 10 μM indexed P7 primer. PCR was run for 18 cycles with the following settings: 72° C. for 5 min, 98° C. for 30 sec, 18 cycles of (98° C. for 10 sec, 66° C. for 30 sec, 72° C. for 30 sec) and a final 72° C. for 5 min. These libraries were then pooled and sequenced on a Nextseq 500 (Illumina, San Diego, Calif.) using a high output 75 cycle kit (Read 1: 18 cycles, Read 2: 52 cycles, Index 1: 10 cycles and Index 2: 10 cycles).

Designing and Fabricating Transfer Clips

Clips were fabricated to securely hold the space-grid slides and tissue slides together during transfer of the oligo hashes from the spotted agarose to the nuclei within the embryo tissue sections. Clips were designed in SolidWorks v24 (Dassault Systemes SolidWorks Corp., Waltham, Mass.). The clips spanned the stacked transfer and tissue slides' width and included fastening features on each end with slight overhangs that fit over the top of the stacked slides. The clips were 3D printed on a Makergear M2 (Makergear, Beachwood, Ohio) printer using consumer grade poly(lactic acid) plastic filament (Makergear). Two clips were used per transfer, placed one on either side of the embryo section. An exemplary clip is shown in FIG. 12.

Transferring Spatially Restricted Oligos

Serial sections of an E14 mouse embryo were purchased (Zyagen, San Diego, Calif.) and stored at −80° C. prior to use. Barcode labels (oligo hashes) were transferred in their arrayed pattern from the substrates (referred herein as space-grid slides) to fresh-frozen embryo sections by diffusion through cell permeabilization buffer. Briefly, the embryo slide was placed so that it rested (tissue facing up) with the tissue section between two transfer clips. Subsequently, 500 μL of cell permeabilization buffer [10 mM Tris/HCl pH 7.4, 10 mM NaCl, 3 mM MgCl2 with 1% v/v superase inhibitor (Invitrogen) and 0.1% v/v IGEPAL CA-630 (Sigma Aldrich)] with 5 μL of slide-specific hashing oligo at 10 μM and 5 μL of 500 μM stock DAPI, was pipetted gently onto the tissue section and across the long edge of the embryo slide nearest the user using an wide bore tip. A space-grid transfer slide was then positioned (agarose surface facing the tissue section) so that the arrayed oligos were aligned between the two transfer clips and spanned the tissue section's extent. Placement of the space-grid slide was achieved by tilting the slide so that its long edge nearest the user contacted the edge of the tissue section slide and fit under the overhanging fastening teeth of the transfer clips. The space-grid slide was then rocked toward the embryo section slide until the two slides were face-to-face with the tissue section contacting the space-grid oligo array-laden agarose membrane. Excess buffer was allowed to wick into a laboratory wipe. When stacked, the slides snapped into the transfer clips and were thereby securely held together during transfer. The slide stack was moved to the microscope stage and the entire embryo section was imaged in GFP and DAPI channels. The transfer slide was then removed from the transfer clips and separated from the tissue. Cells of the embryo section were then scraped using a cell scraper from the slide into a 4% paraformaldehyde fixing solution. After fixation for 15 minutes on ice, cells were spun down in 1.5 mL tubes in a chilled benchtop centrifuge at 800×g for 10 minutes. The supernatant in each tube was removed and cells were pooled in 1 mL of NSB [Nuclei Buffer (10 mM Tris/HCl pH 7.4, 10 mM NaCl, 3 mM MgCl2) with 1% v/v superase inhibitor (Invitrogen) and 1% v/v BSA (New England Biolabs)], flash frozen and stored at −80 C.

Sci-RNA-Seq Library Construction

Frozen nuclei were thawed over ice and spun down at 800 g for 8 minutes in a 15 mL conical. Cells were then permeabilized in permeabilization buffer (NSB+0.25% Triton-X) for 3 minutes and then spun down. Following another wash in NSB, two-level sci-RNA-seq libraries were prepared as previously described. Briefly, nuclei were pelleted at 800×g for 8 minutes and resuspended in 400 μL of NSB. Nuclei were then sonicated for 12 seconds using the bioruptor sonicator on the low setting. Cell counts were obtained by staining nuclei with 0.4% trypan blue (Sigma-Aldrich) and counted using a hemocytometer. 5000 nuclei in 2 μL of NSB and 0.25 μL of 10 mM dNTP mix (Thermo Fisher Scientific, cat no. R0193) were then distributed onto a skirted twin.tec 96 well LoBind plate (Fisher Scientific, cat no. 0030129512) after which 1 μL of uniquely indexed oligo-dT (25 μM) was added to every well, incubated at 55 C for 5 minutes and placed on ice. 1.75 μL of reverse transcription mix (1 μL of Superscript IV first-strand buffer, 0.25 μL of 100 mM DTT, 0.25 μL of Superscript IV and 0.25 μL of RNAseOUT recombinant ribonuclease inhibitor) was then added to every well and plates incubated at 55 C for 10 minutes and placed on ice. Wells were pooled using wide bore tips, and nuclei transferred to a flow cytometry tube through a 0.35 μm filter cap and DAPI added to a final concentration of 3 μM. Pooled nuclei were then sorted on a FACS Aria II cell sorter (BD) at 200 cells per well into 96 well LoBind plates containing 5 μL of EB buffer (Qiagen) and 0.75 μL of second strand mix (0.5 μL of mRNA second strand synthesis buffer and 0.25 μL of mRNA second strand synthesis enzyme, New England Biolabs). Second strand synthesis performed at 16 C for 150 minutes. Tagmentation was then performed by addition of 5.75 μL of tagmentation mix per well (0.01 μL of a custom n7-loaded Tn5 enzyme in 5.74 μL 2×Nextera TD buffer, Illumina) and plates incubated for 5 minutes at 55 C. Reaction was terminated by addition of 12 μL of DNA binding buffer (Zymo) and incubated for 5 minutes at room temperature. 36 μL of Ampure XP beads were added to every well, DNA purified using the standard Ampure XP protocol (Beckman Coulter) eluting with 17 μL of EB buffer and DNA transferred to a new 96 well LoBind plate. For PCR, 2 μL of indexed P5, 2 μL of indexed P7 and 20 μL of NEBNext High-Fidelity master mix (New England Biolabs) were added to each well and PCR performed as follows: 75 C for 3 minutes, 98 C for 30 seconds and 18 cycles of 98 C for 10 seconds, 66 C for 30 seconds and 72 C for 1 minute followed by a final extension at 72 C for 5 minutes. After PCR, all wells were pooled, concentrated using a DNA clean and concentrator kit (Zymo) and purified via a 0.8× Ampure XP cleanup. Final library concentrations were determined by Qubit (Invitrogen), libraries visualized using a TapeStation D1000 DNA Screen tape (Agilent) and libraries sequenced on a Nextseq 500 (Illumina) using a high output 75 cycle kit (Read 1: 18 cycles, Read 2: 52 cycles, Index 1: 10 cycles and Index 2: 10 cycles).

Pre-Processing of Sequencing Data

Sequencing data was processed as described previously (Srivatsan, S. R. et al. Massively multiplex chemical transcriptomics at single-cell resolution. Science 367, 45-51 (2020)). Briefly, sequencing runs were first demultiplexed using bcl2fastq v.2.18. Only barcodes that matched reverse transcription indices within an edit distance of 2 bp were retained. For sci-RNA-seq3 libraries, barcodes which matched both provided reverse transcription indices and ligation indices within an edit distance of 2 bp were retained. Following assignment of indices, polyA tails were trimmed using trim-galore, and reads were mapped to a human transcriptome (hg-38) or human-mouse transcriptome (hg-38 and mm-10) using the STAR aligner. Following alignment, reads were filtered for alignment quality, and duplicates were removed. Reads were considered duplicates if they (1) mapped to the same gene, (2) mapped to the same cell barcode and (3) contained the same unique molecular identifier (UMI). Reads that met the first two criteria and differed by an edit distance of 1 from a previously observed UMI were also marked as duplicates and discarded. Non-duplicate reads were assigned to genes using bedtools to intersect with an annotated gene model. All 3′ UTRs in the gene model were extended by 100 bp to account for the possibility that some gene 3′ UTR annotations may be too short, causing genic reads to improperly be annotated as intergenic. Cell barcodes were considered to correspond to a bona fide cell if the number of unique reads associated with the barcode was greater than an interactively defined threshold on a knee plot. Reads from cells that passed this UMI count threshold were first aggregated into a sparse matrix format and then loaded and saved as a CDS object for analysis with Monocle 3.

Slide Registration

To map cell locations to an image of the embryo, reference regions (fluorescent SYBR green spots) with known positions are used to orient cells. Images of the hash array with fluorescent SYBR green spots on top of the DAPI-stained embryo section were taken with a 2.5× magnification (Zeiss Observer Z1 Microscope). These images were used to orient the hash array to the embryo section. More specifically, co-registration of the imaged embryo sections and the oligo hash tagged transcriptomes was achieved through alignment of the SYBR green waypoints imaged during transfer to their position within an ideal space-grid layout. Coordinates for SYBR green spots imaged during oligo hash transfer to the embryo section and the corresponding coordinates in an image of an ideal space-grid were obtained in Fiji image processing software using the Big Warp function of the BigDataViewer plugin. An affine matrix was computed using the coordinates as source (embryo image) and target (space-grid image) control points in the AffineTransformation function in the “vec2dtransf” and “imager” packages in R. The matrix was applied to the embryo section image. Sequenced nuclei were then mapped to the aligned space of the transformed image and space-grid using their space-grid hashes.

The following formula was used to calculate the number of microns per pixel and therefore estimate the size of each spot on the hash array.

$$microns per 1 pixel = ( native camera pixel size / objective / camera adaptor )$$

One pixel was equal to 1.816 microns based on the camera pixel size of 4.54 for a Zeiss Axiocam 503 Mono Camera, an objective size of 2.5× and a camera adaptor size of 1.

Assigning Spatial Labels from Hash Reads

Reads from hash oligos were demultiplexed as described previously. Briefly, demultiplexed reads that matched combinatorial indexing barcodes were examined to identify hash reads. Reads were considered hash reads when they met two criteria: 1) the first 10 bp of read 2 matched a hash barcode in the experiment within an edit distance of two; and 2) contained a polyA track between base pairs 12 to 16 of read 2. These reads were then deduplicated by cell barcode and collapsed by UMIs to create a vector of hash oligo UMI counts for each nucleus in the experiment.

To assign each nucleus to the slide from which it came, it was tested whether its sci-RNA-seq library was enriched for a particular hash barcode. A nucleus's hash UMIs was compared against a ‘background distribution’, which under ideal circumstances, would be random and uniformly distributed. To estimate the background distribution, the hash UMIs were simply aggregated from cell indices for which fewer than 10<mRNA UMIs were collected, reasoning that these reflect library contributions from ambient reverse transcriptase products, debris fragments, etc. The hash UMIs for nucleus to this background were compared by a chi-squared test. After correcting the resulting p values were corrected for multiple testing by the Benjamini-Hochberg procedure, the null hypothesis that originates from the background distribution at specified FDR (5% FDR was used in this study) was rejected. Those nuclei with hash counts deemed different than background were then evaluated for enrichment for a single hash sequence. Enrichment ratios were calculated as the UMI count ratio of the most abundant vs. the second most abundant hash oligo. Specifically, if the UMI count for the most abundant hash in nucleus is a-fold higher than the second most abundant, is marked as a singleton. a was determined on a per-experiment basis by examining the distribution of these ratios and choosing a value that separated unlabeled cells and singularly labeled cells. Cells that fell below a-fold enrichment of a unique hash oligo were flagged as a multiplet or debris and discarded.

A cell's spatial position within the grid consists of a specific combination of two oligo nucleotides, a spot oligo and a sector oligo. To find a cell's position within the grid, the inventors first took a single cell's vector of sector counts and took its outer product with that cell's vector of spot oligo counts. The product of spot and sector oligos were then ranked and a cell was mapped to the top-ranking combination which matched 2 criteria: (1) the combination represented a valid pairing and (2) the combination mapped within the boundary of the imaged embryo. This boundary was determined by manually segmenting the outline of the DAPI stained image of the embryo.

Determining Nuclei Count from Embryo Images

Using Python, the 2.5× magnification embryo DAPI stained images were preprocessed using a white top-hat transform followed by a histogram equalization to reduce uneven lighting and increase contrast respectively. The images were then thresholded using Otsu's method. In order to overcome the challenge of counting individual cells in nuclei clusters, the resulting binary masks were separated into ‘dense’ and ‘sparse’ nuclei masks using a connected components algorithm. The dense nuclei masks were used to isolate nuclei clusters in the original embryo images, which were then thresholded to a secondary value defined as Otsu's value plus a constant intensity shift. The sparse and the dense nuclei masks were then distance transformed, using Euclidean geometry, to generate distance maps. A peak finding algorithm was used to isolate the centroids of peaks in the distance maps and resulting unique centroids were counted as nuclei.

Cell Type Classification

Nearest neighbor classification was performed by aligning the cells from this study to cells from E13.5 time point from the MOCA single cell dataset. Count matrices from the two datasets were subsetted for genes found in both datasets and then combined. The E13.5 time point was then downsampled and the two datasets were aligned using the mutual nearest neighbor algorithm with the function align_cds( ) in Monocle3. For each cell in the E14 time point the 5 nearest neighbors from the MOCA dataset were recorded. The procedure of sampling and aligning the datasets was repeated 15 times. Each cell was then assigned the majority nearest neighbor label over the 15 trials. Finally, to remove poor confidence cell type labels, the E14 data was clustered and cell type labels that did not account for more than 5% of a cluster were assigned “Unknown”. Garnett classification was performed using a marker-free classifier trained on the E13.5 time point from the MOCA dataset. This classifier was then applied to the cells sequenced in this study.

Immunostaining and Adjacent Image Alignment

Before immunostaining serial sections adjacent to sequenced sections were fixed in 4% paraformaldehyde (Electron Microscopy Sciences) in PBS for three minutes at room temperature. Sections were then washed for five minutes three times in PBS-T (0.1% Tween-20, VWR), permeabilized for 10 minutes at room temperature in 0.1% Triton X-100 (VWR) in PBS and washed for five minutes three times in PBS. Autofluorescence was quenched using TrueBlack Lipofuscin (Biotium) according to the manufacturer's protocol. Briefly, sections were treated with TrueBlack Lipofuscin diluted 20× in 70% ethanol with a 30 second to three-minute incubation, then washed for five minutes three times with PBS. Sections were next blocked with 2.5% normal donkey serum (NDS, Jackson ImmunoResearch Laboratories) in PBS for one hour at room temperature. Primary antibodies were applied in PBS containing 2.5% NDS as indicated in Table 1 with an overnight incubation at 4° C. The following day sections were washed for five minutes three times in 2.5% NDS in PBS, then incubated for one hour at room temperature with Hoechst 33342, Trihydrochloride, Trihydrate (Invitrogen) counterstain and secondary antibodies diluted as indicated in Table 1 in 2.5% NDS in PBS. Sections were next washed again in 2.5% NDS in PBS and then coverslipped with Fluoromount-G Mounting Medium (SouthernBiotech) prior to imaging.

Stained sections were imaged using a Ti-E inverted microscope (Nikon) and multi-field images were stitched in the NIS-Elements (Nikon) software. Each channel of the triple stained sections together with the counterstain was aligned to the DAPI image of the sequenced section using the StackReg plugin in Fiji (rigid body followed by affine). Each aligned channel of the stained section images were then separated from the counterstain and overlaid onto the DAPI channel image of the adjacent sequenced section.

Anatomical Annotation and Segmentation

Annotation was performed using The Atlas of Mouse Development in conjunction with magnetic resonance images of the E14.5 embryo. Annotations were then confirmed using immunostained adjacent sections (when available). Anatomical segmentation was performed manually using the DAPI-stained embryo section and the Big Warp function of the BigDataViewer plugin in Fiji. The region of interest was demarcated by choosing a bounding set of points in clockwise or counter-clockwise order. These points were then used to construct a polygon using the spatial features package in R. These polygons were then scaled using the same affine transformation used for slide registration to put them on the same coordinate axis. For polygons with holes, the contour of the entire image was first segmented followed by segmentation of each cavity.

Kriging Gene Expression

Spatial grid positions were first collapsed such that non-overlapping sets of 4 adjacent positions were collapsed into a single spatial position. Each cell type within these spatial bins was then aggregated by summing the counts for each gene contributed by that cell type. These values were then kriged with the automap package in R using ordinary kriging via the autoKrige( ) function. Interpolated values were then rescaled to reflect the percentage of gene expression contributed by a cell type at each given position. Finally, a polygon object specific to each slide was used to clip the interpolated gene expression values.

Spatial Autocorrelation Analysis

Gene spatial autocorrelation was computed by first subsetting cell types for which there were more than 50 cells present on a slide. Then for each cell type and each slide, a gene's spatial autocorrelation was computed using a cell's spatial coordinates as the input into Monocle3's graph_test( ) function. The resulting test statistic was corrected for multiple testing and genes with an FDR<0.01 and a Moran's I test statistic greater than 0.05 were reported as having statistically significant spatial autocorrelation.

Variance Decomposition Model

The variance of gene expression within and between cell populations was computed using the angular distance metric. Specifically, let y=the vector of log-scaled gene expression levels for each gene in a cell, normalized to be of unit magnitude, and let Y=the empirical distribution of y across all cells in a given analysis. Then:

$$Var ( Y ) := E [ angular\_distance ( Y - E [ Y ] ) \hat{} 2 ]$$

$$where angular\_distance ( a , b ) = ( 2 / π ) {cos}^{- 1} ( ( A \cdot B ) / ( ‖ A ‖ ‖ B ‖ ) )$$

This variance statistic behaves similarly to the variance of a univariate distribution. Most importantly, it obeys the Law of Total Variance. If cells sampled from Y are partitioned into groups X, then:

$$Var ( Y ) = E [ Var ( Y \vert X ) ] + Var ( E [ Y \vert X ) ] ) == weighted average of within \text{-} group variance + weighted average of between \text{-} group variance$$

The “variance explained” by a grouping X, e.g. grouping cells by cell type, can therefore be computed as:

$$Variance explained = 1 - \frac{within \text{-} group var .}{total var .} = 1 - \frac{E [ Var ( Y \vert X ) ]}{Var ( Y )}$$

This naive formula for variance explained is strongly affected by the number of groups in the grouping X, similar to how the variance explained in a regression model is affected by the number of degrees of freedom in the model. If one divides a population of cells into random groups of two, variance explained will be high, even though the grouping has no biological meaning. It can be correct for this by using an adjusted formula:

$$Variance explained = 1 - \frac{E [ Var ( Y \vert X ) ]}{E [ Var ( Y ) \vert permuted X ) ]}$$

where in the denominator, the group id associated with each cell is randomly permuted.

This adjusted formula corrects for the number of groups in X, but is confounded by another factor. The observed variance between cell gene expression vectors is a result of both biological heterogeneity and technical factors such as the sparsity of the single cell RNA-seq data. If the same biological sample is profiled in two different experiments, and the median number of UMIs per cell is N in experiment 1 and 4N in experiment 2, then the variance explained by the grouping X=cell type will be lower in experiment 1 vs. 2 due to increased sparsity. To correct for sparsity and estimate the proportion of biological variance explained by grouping X, a final adjusted formula was used:

$$Variance explained = 1 - \frac{E [ Var ( Y \vert X ) ] - E [ Var ( resampled Y \vert X ) ]}{E [ Var ( Y \vert permuted X ) ] - E [ Var ( resampled Y \vert permuted X ) ]}$$

In this formula, the “resampled Y” terms are computed by replacing each cell's gene expression vector y with a sample from the distribution Multinomial(n=# of UMIs for the cell, p=E[Y|X]); in other words, resampling from the group means. Resampling in this manner is a way of estimating what variance one would observe if there were no biological heterogeneity in a group of cells and the only source of observed heterogeneity was sparsity.

This formula was applied to estimate the proportion of biological gene expression variance explained by cell type; the proportion of biological gene expression variance explained by spatial position, using the grouping X=a spatial spatial bin (non-overlapping 2*2 spot squares); the proportion of biological gene expression variance explained by spatial position and cell type where X=the combination of spatial bin and cell type. FIG. 4B shows application of this formula to estimate the proportion of the residual biological gene expression variance, after accounting for cell type, that is explained by spatial position. In these models, the grouping X=the tuple (cell type, spot id), and permuted X=the tuple (cell type, permuted spot id). By permuting the spot id but not the cell type annotation, it is ensured that the estimate is of the variance explained by the cell type+space model relative to the cell-type-only model, rather than relative to a null model.

Pairwise Angular Distance

Only cell types with 100 cells or more, originating from a single slide were considered for this analysis. First, 5000 random pairings between cells of the same type and from the same slide were tabulated along with the euclidean distance between the two cells. Next each cell was size factor normalized and scaled to the unit hypersphere. The pairwise angular distance was then calculated as detailed above. To test whether there was a relationship between angular distance and physical distance, a linear model was fit with angular distance as the response and physical distance as the sole predictor. Reported p values indicate the significance of coefficient for the physical distance predictor variable.

Spatial Gene Modules

Spatial autocorrelation was calculated using endothelial cells from every section using the sci-space derived spatial coordinates as the input into Monocle3's graph_test( ) function. Spatially autocorrelated genes were defined as those genes which exhibited a Moran's I value greater than 0.05, were below a FDR cutoff of 0.001 and expressed in at least 1% of cells. These spatially autocorrelated endothelial genes were then used to subset endothelial cells and as subsequent input into Monocle3's find_gene_modules( ) function. Gene expression for each cell from Slide 1 was then aggregated by genes in each gene module and then kriged with the automap package in R using ordinary kriging via the autoKrige( ) function. Gene Ontology (GO) enrichment analysis was then performed for genes from each module using a GO enrichment webtool (http://geneontology.org). These endothelial cell gene modules were also used to aggregate gene expression in an aligned co-embedding of the sci-space and MOCA datasets.

TABLE 1
Reagents and dilutions for immunostaining

| Host, Target or Counterstain | Vendor, Catalog # | Dilution |
|---|---|---|
| Goat, E-Cadherin | R&D Systems, AF748 | 1:100 |
| Rabbit, Sarcomeric Alpha-Actinin | Abcam, ab68167 | 1:100 |
| Mouse, MYH6 | Abcam, ab50967 | 1:200 |
| Donkey, Goat IgG | ThermoFisher, A11055 | 1:1000 |
| Donkey, Rabbit IgG | ThermoFisher, A31573 | 1:1000 |
| Donkey, Mouse IgG | ThermoFisher, A31570 | 1:1000 |
| Hoechst 33342, Trihydrochloride, | Invitrogen, H3570 | 1:2000 |
| Trihydrate | | |

While illustrative embodiments have been illustrated and described, it will be appreciated that various changes can be made therein without departing from the spirit and scope of the invention.

1. A substrate for spatially barcoding a tissue sample, comprising a matrix, wherein the matrix comprises:
(a) a plurality of discrete labeling regions arranged in a pattern, wherein the labeling regions are separated by non-labeling regions, and wherein each labeling region comprises one or more barcode labels, wherein the one or more barcode labels can be transferred from the matrix into a tissue sample upon contacting the substrate with the tissue sample; and
(b) a support.

2. The substrate of claim 1, wherein the matrix further comprises a plurality of discrete reference regions, wherein each reference region comprises one or more reference labels, wherein the one or more reference labels can be transferred from the matrix into a tissue sample upon contacting the substrate with the tissue sample.

3. (canceled)

4. The substrate of claim 1, wherein the matrix comprises a natural or synthetic polymer.

5. (canceled)

6. The substrate of claim 1, wherein the one or more barcode labels are fluorescent or non-fluorescent.

7. The substrate of claim 2, wherein the one or more reference labels comprise one or more fluorescent dyes and/or a staining dye.

8-10. (canceled)

9. The substrate of claim 1, wherein the one or more barcode labels comprise a molecular tag indicating spatial coordinates within the matrix.

10-13. (canceled)

11. The substrate of claim 1, wherein each labeling region comprises a unique combination of the one or more barcode labels.

12. The substrate of claim 2, wherein the labeling regions and/or reference regions are prepared by spotting droplets of a solution comprising the one or more barcode labels onto the matrix.

13. (canceled)

14. The substrate of claim 1, wherein the labeling regions have a distance of about 250 μm, about 220 μm, about 200 μm, about 190 μm, about 180 μm, about 170 μm, about 160 μm, about 150 μm, about 140 μm, about 130 μm, about 120 μm, about 110 μm, about 100 μm, or about 50 μm between the centers of the labeling regions.

15. The substrate of claim 1, wherein the labeling regions have a diameter of from 0.5 μm to 25 μm, from 1 μm to 200 μm, or from 50 μm to 200 μm.

16. The substrate of claim 1, wherein the labeling regions are arranged in a grid.

17. The substrate of claim 1, wherein the substrate comprises about 3,000, about 4,000, about 5,000, about 6,000, about 7,000, about 8,000, about 9,000, about 10,000, about 25,000, about 50,000, about 100,000, about 200,000, about 300,000, about 400,000, about 500,000, about 600,000, about 700,000, about 800,000, about 900,000, or about 1,000,000 unique labeling regions in a 10 mm by 10 mm area.

21. (canceled)

22. The substrate of claim 1, wherein the support comprises glass or synthetic polymer.

23. (canceled)

24. A method of spatially labeling cells in a tissue sample, comprising contacting a tissue sample comprising a plurality of cells with the substrate of claim 1 for a time sufficient for at least a portion of the one or more barcode labels to transfer into at least a portion of the plurality of cells.

25-28. (canceled)

29. The method of claim 24, wherein the method further comprises dissociating the tissue sample into individual cells after contacting the sample with the substrate.

30. The method of claim 24, wherein the tissue sample is sandwiched between a glass slide or plastic slide and the substrate during the contacting.

31. A kit for spatially labeling cells in a multi-cellular sample, comprising the substrate of claim 1.

32. (canceled)

33. A method of spatial gene expression profiling in a multi-cellular sample, comprising:
(a) contacting a tissue sample comprising a plurality of cells with the substrate of claim 2 for a time sufficient for at least a portion of the one or more barcode labels to transfer into at least a portion of the plurality of cells;
(b) imaging the substrate to determine positions of the reference regions;
(c) extracting nuclei from each cell; and
(d) analyzing gene expression in each individual cell.

34. The method of claim 33, wherein the method comprises dissociating the tissue sample into individual cells after imaging the substrate.

35. (canceled)

36. The method of claim 33, wherein the tissue sample is sandwiched between a glass slide or plastic slide and the substrate during the contacting.

37. (canceled)
